(19) United States
(12) Patent Application Publication
(10) Pub. No.: US 2013/0157924 A1
(43) Pub. Date: Jun. 20, 2013

(54) REDUCING TRANSMISSION OF SEXUALLY TRANSMITTED INFECTIONS

(71) Applicants: Stephen Dewhurst, Rochester, NY (US); Bradley Nilsson, Rochester, NY (US); Joanna Olsen, Rochester, NY (US); Jerry Yang, LaJolla, CA (US)

(72) Inventors: Stephen Dewhurst, Rochester, NY (US); Bradley Nilsson, Rochester, NY (US); Joanna Olsen, Rochester, NY (US); Jerry Yang, LaJolla, CA (US)

(73) Assignees: THE REGENTS OF THE UNIVERSITY OF CALIFORNIA, Oakland, CA (US); University of Rochester, Rochester, NY (US)

(21) Appl. No.: 13/643,026

(22) PCT Filed: Apr. 22, 2011
(86) PCT No.: PCT/US11/33659

§ 371 Date: Mar. 6, 2013

Related U.S. Application Data
(60) Provisional application No. 61/327,461, filed on Apr. 23, 2010.

(51) Int. Cl. A61K 31/428; A61K 47/48; A61K 45/06

(52) U.S. Cl. CPC A61K 31/428; A61K 45/06; A61K 47/48238
USPC 514/1.1; 514/367; 514/34; 514/50

(57) ABSTRACT

Described herein are compositions and methods for treating or preventing a sexually transmitted infection in a subject.

### STATEMENT REGARDING FEDERALLY FUNDED RESEARCH

This invention was made with government support under Grant Nos. R21 AI074351, RO1AI084111, and T32 DA07232 from the National Institutes of Health. The United States government has certain rights in this invention.

### BACKGROUND

There are over twenty types of sexually transmitted infections (STI) associated with various bacteria, protozoa, fungi and viruses. One example of a viral sexually transmitted infection is human immunodeficiency virus (HIV). Acquired immunodeficiency syndrome (AIDS) is a collection of symptoms and infections resulting from the specific damage to the immune system caused by HIV. In 2006, nearly 25 years after the first report of AIDS cases, there were over 39 million persons living with HIV infection worldwide. About one-fourth of those with infected with HIV have not yet been diagnosed and are unaware of their status. AIDS has become one of the deadliest epidemics in human history, killing more than 25 million people around the world. In the last decade, major advances in prevention and treatment for HIV/AIDS have prolonged and improved the lives of many, but despite such advances, an estimated 4 million people still become infected with HIV every year, and many of these are people under the age of 25. In 2006, HIV/AIDS was responsible for nearly 3 million deaths worldwide.

### SUMMARY

Provided herein are methods of treating or preventing a sexually transmitted infection in a subject. The methods comprise administering to a subject with or at risk of acquiring a sexually transmitted infection a semen-derived enhancer of viral infection (SEVI)-binding agent comprising a compound described herein, including, e.g., BTA-EG₄ and BTA-EG₆. Also provided are methods comprising administering to a subject with or at risk of acquiring a sexually transmitted infection a semen-derived enhancer of viral infection (SEVI)-binding small molecule. The SEVI-binding small molecule can, for example, comprise a hydrophobic molecule that incorporates into or binds the SEVI-fibrils or an anionic polypeptide supramolecular assembly. The methods can further comprise administering to the subject an anti-viral, an anti-bacterial, or an anti-fungal agent.

Also provided are pharmaceutical compositions comprising a first agent, which is a semen-derived enhancer of viral infection (SEVI)-binding agent or small molecule (e.g., a hydrophobic molecule that incorporates into ro binds the SEVI-fibrils or an anionic polypeptide supramolecular assembly) as described herein, and a second selected from the group consisting of an anti-viral, an anti-bacterial, and an anti-fungal agent.

The details of one or more implementations are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B are graphs showing semen-derived amyloid fibrils referred to as SEVI (semen-derived enhancer of virus infection) stimulates inflammatory cytokine production by primary human macrophages. FIG. 1A is a graph showing IL-1θ, levels and FIG. 1B is a graph showing TNFI levels in primary human macrophages treated with SEVI or mock treated.

FIGS. 2A and 2B show a schematic of a potential mechanism of a microbicide against SEVI (FIG. 2A) and SEVI-binding molecules (2B).

FIGS. 3A and 3B show that prostatic acid phosphatase (PAP) 248-286 forms fibrils. PAP248-286 (10 mg/ml in PBS) was agitated at 37° C. and 14,000 RPM. FIG. 3A is a graph showing the results of samples collected at 0 hours, 24 hours, 48 hours, 72 hours, and 96 hours that were subjected to Thioflavin T analysis. FIG. 3B shows images of SEVI-fibrils visualized by electron microscopy. Samples were collected at 72 hours.

FIGS. 4A and 4B show that SEVI fibrils enhance HIV-1 infection in vitro. 5×10⁴ CEM 5.25 cells were exposed to infectious HIV-1 (pNL43; 1.2 ng of virus, as determined by p24 ELISA assay) for 2 hours in the presence (10 or 25 μg/mL) or absence of SEVI. FIG. 4A is a graph showing luciferase activity measured in cell lysates at 48 hours post infection. FIG. 4B shows fluorescence microscopy images of GFP at 48 hours.

FIGS. 5A and 5B show that the Thioflavin-T analogs BTA-EG₄ and BTA-EG₆ inhibit SEVI mediated enhancement of HIV infection. CEM 5.25 cells were exposed to infectious HIV-1 (IIIB) for 2 hours in the absence or presence (25 μg/mL) of SEVI fibrils; BTA-EG₄ (FIG. 5A) and EG₆ (FIG. 5B) was added at concentrations of 5.5, 11 and 16.5 μg/mL. Luciferase activity was measured in cell lysates 72 hrs post-infection. * indicates p value<0.05.

FIGS. 6A and 6B show BTA-EG₄ and BTA-EG₆ inhibit semen mediated enhancement of HIV Infection. HIV-1 IIIB virions were preincubated with 50% semen, with or without increasing concentrations of BTA-EG₄ (FIG. 6A) and BTA-EG₆ (FIG. 6B). After 10 minutes these stocks were diluted 15 fold into CEM 5.25 cells. Cells were washed after 1 hour and luciferase expression was measured at 48 hours to quantify the extent of infection. * indicates p value<0.05.

FIGS. 7A and 7B show BTA-EG₄ and BTA-EG₆ decrease SEVI enhanced binding of HIV to target cells. HIV-1 (IIIB) virions were pretreated with 10 ug/mL SEVI and added to Jurkat cells with or without increasing concentrations of BTA-EG₄ (FIG. 7A) or BTA-EG₆ (FIG. 7B). After 90 minutes, cells were washed to remove any unbound virus and bound virions were detected using a p24 ELISA.

FIG. 8 shows fluorescence polarization analysis of heparin binding to SEVI fibrils. SEVI was diluted to concentrations ranging from 5 to 100 μg/ml, in the presence of 16 μg/ml of FITC-heparin. Samples were incubated 1 hour at RT, and read at excitation λ=480, and emission λ=535. The graphs show the competitive displacement of bound FITC-heparin from SEVI fibrils. SEVI (100 μg/ml) and FITC-heparin (16 μg/ml) were combined as demonstrated in the left graph. In the right graph, unlabeled heparin was then added, in serial 10-fold dilutions from 3000 to 3 μg/ml.

FIGS. 9A and 9B show that fluorescence polarization detects binding of BTA-EG₄ (FIG. 9A) and BTA-EG₆ (FIG. 9B) to SEVI fibrils. 100 ug/mL of SEVI was mixed with 16 ug/mL FITC-heparin in varying concentrations of BTA-EG₄ or BTA-EG₆. Samples were incubated 1 hour at room temperature and polarized fluorescence intensities were measured.

FIGS. 10A and 10B show BTA-EG₄ and EG₆ are not toxic to cervical epithelial Cells. The cervical epithelial cell lines A2En (endocervical) (FIG. 10A), 3EC1 and SiHa (FIG. 10B) were treated for 12 hours with BTA-EG₄ and BTA-EG₆ at concentrations up to 10 times greater than the inhibitory concentration. At 12 hours, viability was measured with Alamar Blue.

FIGS. 11A and 11B show BTA-EG₄ and BTA-EG₆ do not induce cytokine production in cervical epithelial cells. A2En, 3EC1 and SiHa Cells were treated with BTA-EG4 or BTA-EG6 at varying concentrations for 6 hours. At 6 hours, supernatants were collected and cytokine production (IL-1β (FIG. 11A); Mip3α (FIG. 11B); and TNF-α) was determined by ELISA. Representative results from Siha cells are shown.

FIG. 12 shows that the thioflavin-T analog BTA-EG₆ binds SEVI fibrils. In FIG. 12A shows the chemical structure of ThT and BTA-EG₆. FIG. 12B shows that BTA-EG₆ binds SEVI fibrils as measured by fluorescence polarization. 100 μg/ml SEVI was mixed with 16 μg/ml FITC-heparin in varying concentrations of BTA-EG₆ ranging from 0 to 200 μg/ml. Samples were incubated 1 h at room temperature, and polarized fluorescence intensities were measured. Decreased millipolarization units (mP) indicate a displacement of FITC-heparin from SEVI fibrils due to BTA binding. FIG. 12C shows binding of BTA-EG₆ to SEVI fibrils as determined by a centrifugation assay. Briefly, various concentrations of BTA-EG₆ in PBS were incubated overnight at room temperature in the presence or absence of SEVI fibrils. After equilibration, each solution was centrifuged, and the supernatants were separated from the pelleted fibrils. The fluorescence of BTA-EG₆ was determined from the resuspended pellets in PBS solution. Error bars represent ±S.D. of duplicate measurements. The Kd was determined by fitting the data to a one-site specific binding algorithm: Y=Bₘₐₓ×X/(Kd+X), where X is the concentration of BTA-EG₆, Y is the specific binding fluorescence intensity, and Bₘₐₓ corresponds to the apparent maximal observable fluorescence upon binding of BTA-EG₆ to SEVI fibrils. RFI, relative fluorescence intensity. FIG. 12D shows that BTA-EG₆ does not affect the stability of SEVI fibrils. Preformed SEVI fibrils were incubated with increasing concentrations of BTA-EG₆ for 3 h. Fibril stability was measured by ThT fluorescence. FIG. 12E shows that BTA-EG₆ binding to SEVI inhibits the interaction of SEVI fibrils with the cell surface. Jurkat T cells were incubated with SEVI-biotin for 1 h in the presence or absence of 5.5 μg/ml (low) or 27 μg/ml (high) BTA-EG₆. Surface-bound fibrils were detected with SA-FITC and measured by flow cytometry. Results are summarized in Table 1 and are representative of three experiments that were performed with similar results.

FIG. 13 shows that BTA-EG₆ inhibits SEVI-mediated enhancement of HIV-1 infection. In FIG. 13A, HIV-1 IIIB virions were preincubated with increasing concentrations of BTA-EG₆ (0, 5.5, 11, and 22.5 μg/ml) and with or without SEVI (15 μg/ml) as indicated. The samples were then added to CEM-M7 cells. Cells were washed at 2 h, and infection was assayed at 48 h by measuring Tat-driven luciferase expression. Results shown are average values±S.D. of triplicate measurements from one of four independent experiments that yielded equivalent results. * indicates p<0.05 when compared with control cells exposed to HIV-1_{IIIB}+SEVI alone by ANOVA with Tukey's post test. RLU, relative luciferase units; Uninf, uninfected. FIG. 13B is a zoom in of panel A to show data for cells treated with HIV-IIIB virions with and without increasing concentrations of BTA-EG₆, in the absence of SEVI. BTA-EG₆ had no effect on the infectivity of HIV alone; concentrations of BTA-EG₆ are noted above for panel A. FIG. 13C shows the results of CEM-M7 cells infected with HIV-1_{ADA}, as in panel A. FIG. 13D shows that CEM-M7 cells were infected with HIV-1_{ADA}+SEVI with concentrations of BTA-EG₆ ranging from 0.4 to 50 μg/ml. An exponential decay curve was then fit to the data and used to calculate the IC₅₀ of the inhibitory effect of BTA-EG₆ on SEVI-mediated enhancement of HIV-1 infection. In FIG. 13E, human PBMCs were stimulated with IL-2/PHA and infected with HIV-1_{BAL} and increasing concentrations of BTA-EG₆ (0, 5.5, 11, and 22.5_g/ml) with and without SEVI (15 μg/ml). Cells were washed at 3 h, and infection was assayed at 4 days by measuring p24. Results shown are average values±S.D. of triplicate measurements. * indicates p_{—}0.01 when compared with control PBMCs exposed to HIV-1ADA_SEVI alone (ANOVA with Tukey's post test).

FIG. 14 shows that BTA-EG₆ inhibits semen-mediated enhancement of HIV-1 infectivity. FIG. 14A shows HIV-1_{IIIB} virions were preincubated with 50% pooled human semen, with or without increasing concentrations of BTA-EG₆ (5.5, 11, and 22.5 μg/ml). After 10 min, these stocks were diluted 15-fold into CEM-M7 cells. Cells were washed after 1 h, and luciferase expression was measured at 48 h to quantify the extent of infection. Results shown are average values±S.D. of triplicate measurements from one of three independent experiments that yielded equivalent results. * indicates p<0.05 when compared with control cells exposed to HIV-1_{IIIB}+semen alone, by ANOVA with Tukey's post test. RLU, relative luciferase units. In FIG. 14B, cells were treated as above but with HIV-1_{ADA} and a 50% concentration of an individual semen sample. *, p<0.05 when compared with control cells exposed to HIV-1_{ADA}+semen alone, by ANOVA with Tukey's post test. FIGS. 14C and D show that BTA-EG6 does not inhibit semen-mediated cytokine release. SiHa cells were treated with pooled human semen for 6 h, with and without 27 μg/ml BTAEG₆. At 6 h, IL-8 (C) and MIP-3α (D) production in the supernatants was measured by ELISA. Results shown are average values±S.D. of triplicate measurements from one of three independent experiments that yielded equivalent results. N.S=not significant when compared with cells treated with semen alone (as determined by ANOVA with Tukey's post test).

FIG. 15 shows that BTA-EG₆ inhibits SEVI-mediated attachment of HIV-1 to the cell surface. In FIG. 15A, HIV-1_{IIIB} virions were pretreated with or without 10 μg/ml SEVI and added to Jurkat cells with or without increasing concentrations of BTA-EG₆ (5.5, 11, and 22.5 μg/ml). After 90 min, cells were washed to remove any unbound virus, and bound virions were detected using a p24 ELISA. The data show that BTA-EG₆ efficiently inhibited SEVI-mediated enhancement of HIV-1_{IIIB} attachment to Jurkat cells (* indicates p<0.01 for cells treated with SEVI plus 5.5, 11, or 22.5 μg/ml BTA-EG₆ versus cells treated with SEVI alone; ANOVA with Tukey's post test). BTA-EG₆ had no effect on the binding of HIV-1 virions alone to cells. Uninf, uninfected. In FIG. 15B, Jurkat cells were treated as above using HIV-1_{ADA} (* indicates p<0.01 for cells treated with SEVI plus 11 or 22.5 μg/ml BTA-EG₆ versus cells treated with SEVI alone; ANOVA with Tukey's post test). In FIG. 15C, A2En cells were incubated with HIV-1_{ADA} in the presence or absence of 22.5_g/ml BTA-EG₆ (* indicates p<0.01 for cells treated with SEVI plus 22.5 μg/ml BTA-EG₆ versus cells treated with SEVI alone; ANOVA with Tukey's post test). In FIGS. 15A-C, all results shown are average values±S.D. of triplicate measurements from one of three independent experiments that yielded equivalent results. In FIG. 15D, A2En cells were treated with HIV-1BaL and 15 μg/ml SEVI with or without increasing concentrations of BTA-EG₆ (5.5, 11, and 22.5 μg/ml). At 24 h, supernatants were collected and analyzed by ELISA for the presence of IL-8. (* indicates p<0.01 for cells treated with SEVI plus 11 or 22.5 μg/ml BTA-EG₆ versus cells treated with SEVI alone; ANOVA with Tukey's post test).

FIG. 16 shows that BTA-EG₆ is not toxic to cervical cells. In FIG. 16A, the cervical endothelial cell lines A2En (endocervical), 3EC1 (ectocervical), and SiHa were treated for 12 h with BTA-EG₆ at concentrations up to 10 times greater than the IC₅₀. Control cultures were treated with nonoxynol-9 (non-9) at 0.1% final concentration as a positive control for induction of cell death. At 12 h, viability was measured by resazurin cytotoxicity assay (AlamarBlue assay). Representative results from A2En cells are shown; results from 3EC1 and SiHa cells were very similar. In FIGS. 16B and C, BTA-EG₆ does not induce inflammatory chemokine production in cervical epithelial cells. A2En, 3EC1, and SiHa Cells were treated with BTA-EG₆ at varying concentrations for 6 h; control cultures were treated with a well defined TLR2/6 agonist, FSL1 (a synthetic diacylated lipoprotein derived from M. salivarium) at 0.1 μg/ml final concentration as a positive control for chemokine induction. At 6 h, supernatants were collected, and production of Mip-3α (B) and IL-8 (C) was determined by ELISA. Representative results from A2En cells are shown; results from 3EC1 and SiHa cells were very similar. In FIGS. 16A-C, all results shown are average values±S.D. of triplicate measurements from one of three independent experiments that yielded equivalent results. No significant difference (p>0.05) was noted between control cultures treated with PBS and those treated with the highest dose (66 μg/ml) of BTA-EG₆, as determined by ANOVA with Tukey's post test.

FIGS. 17A and B show levels of bound virons using an HIV-1 p24 antigen capture assay with HIV-1 IIIB virions pretreated with 15 μg/ml SEVI and added to 5×10⁴ A2En cells (immortalized primary human endocervical cells) (A) or to Jurkat T cells (a CD4+ human T cell line) (B) in the presence or absence of test compounds (at a final concentration of 25 μM).

FIG. 18A shows a schematic of binding of an amyloid-binding ligand, like benzothiazole aniline (BTA), in monomeric (left panel) or oligomeric (right panel) form. FIG. 18B shows the structure of a benzothiazole aniline (BTA)-based monomer (1), dimer (2), trimer (3), tetramer (4), and pentamer (5). The structure of BTA moiety is given and is represented as simple red ovals in molecules 1-5 for clarity.

### DETAILED DESCRIPTION

The majority of sexually transmitted infections are acquired through unprotected sexual relations, that is, sexual intercourse in the absence of a barrier such as a condom. For example, sexual transmission of HIV can occur when HIV-containing secretions, e.g., seminal or vaginal fluid, of one partner come into contact with the genital, oral, or rectal mucous membranes of another. The epithelial cells of the mucous membranes act, at least in part, as a barrier to viral penetration. HIV can cross the epithelial barrier either by capture by intra-epithelial dendritic cells that convey the virus to target cells deeper in the mucosa or through regions of damaged epithelium resulting from traumatic injury or lesions caused by sexually transmitted diseases. Once the virus has breached the epithelial membrane, the infection spreads among cells of the immune system, including, for example, CD4+ T cells, macrophages and dendritic cells. Ultimately, the virus disseminates via the lymphatic system and the blood to spleen, brain, liver, and lungs. The efficiency of sexual transmission of HIV depends on many factors, including, for example, host factors in both the transmitting partner and the recipient. Seminal fluid contains a number of factors, for example, semen fibrils, amines such as spermine, spermidine, putrescine and cadavarine, as well as nutrients and enzymes that protect the virus from the acidic environment of the vaginal tract and that enhance sexual transmission of HIV.

Cationic polymers enhance retrovirus transduction by neutralizing the electrostatic repulsion between the virus and cell surface and allowing many virus particles to aggregate onto a single surface enhancing the effective multiplicity of infection. As described herein, semen fibrils (e.g., prostatic acid phosphatase (PAP) fibrils) work in a similar manner since semen fibrils are highly cationic. As described herein, and without meaning to be limited by theory, interfering with the binding of infectious agents such as viruses to semen fibrils reduces the risk of sexually transmitted infections. Immunization against semen-derived amyloid fibrils or precursor forms of such fibrils (e.g., peptide oligomers) will not result in autoimmune reactions against wild-type PAP since the PAP-derived amyloid fibrils and their precursor molecules possess unique conformational attributes that distinguish them from the native PAP protein. Further, PAP has been shown to be a safe vaccine antigen in the context of immunization for prostate cancer. Thus, immunization with short linear peptides derived from PAP is safe.

An amyloid-binding small molecule is an efficient inhibitor of SEVI- and semen-mediated enhancement of HIV infectivity. BTA-EG₆ binds to the SEVI fibrils and interferes with their ability to enhance HIV infectivity. Importantly, BTA-EG₆ did not have any direct inhibitory effects on the infectivity of HIV-1 alone.

Provided herein are methods of treating or preventing a sexually transmitted infection in a subject. The methods, optionally, comprise identifying a subject with or at risk of developing a sexually transmitted infection and administering to the subject a semen-derived enhancer of viral infection (SEVI)-binding agent, wherein the agent comprises a compound represented by Formula I:

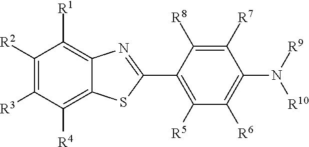

and pharmaceutically acceptable salts and prodrugs thereof. The agent can, for example, bind and prevent the ability of SEVI-fibrils or prefibrillar forms of SEVI from enhancing a sexually transmitted infection in the subject. Optionally, the methods further comprise administering to the subject an anti-viral, an anti-bacterial, or an anti-fungal agent.

In Formula I, R¹, R², R³, R⁴, R⁵, R⁶, R⁷, and R⁸ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl. In some examples, R³ is methyl and R¹, R², R⁴, R⁵, R⁶, R⁷, and R⁸ are hydrogen.

Also, in Formula I, R⁹ and R¹⁰ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, or substituted or unsubstituted heteroaryl. R⁹ and R¹⁰ can combine with the adjacent N to form a heterocycle. In some examples, R¹⁰ is hydrogen. In some examples, R⁹ is the Structure I-A:

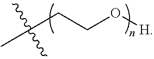

In Structure I-A, n is an integer from 0 to 20. In some examples of Structure I-A, n is 4 or 6.

In Formula I, adjacent R groups on the phenyl ring (i.e., R¹, R², R³, and R⁴; R⁵ and R⁶; and R⁷ and R⁸) can be combined to form substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl groups. For example, R¹ can be a formamide group and R² can be an ethylene group that combine to form a pyridinone group. Other adjacent R groups include the combinations of R² and R³, R³ and R⁴, R⁵ and R⁶, and R⁷ and R⁸.

Specific examples of Formula I are as follows:

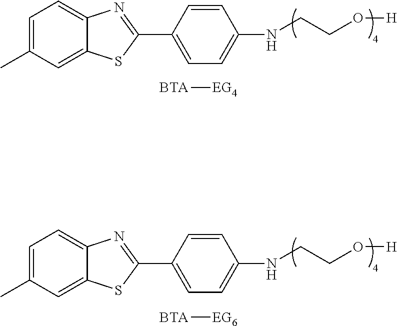

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, pyrazole, and pyrazine. Aryl and heteroaryl molecules can also include additional fused rings, for example, benzofuran, indole, benzothiophene, naphthalene, anthracene, and quinoline.

The alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl molecules used herein can be substituted or unsubstituted. As used herein, the term substituted includes the addition of an alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl group to a position attached to the main chain of the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl, e.g., the replacement of a hydrogen by one of these molecules. Examples of substitution groups include, but are not limited to, hydroxyl, halogen (e.g., F, Br, Cl, or I), and carboxyl groups. Conversely, as used herein, the term unsubstituted indicates the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl has a full complement of hydrogens, i.e., commensurate with its saturation level, with no substitutions, e.g., linear decane (—(CH₂)₉—CH₃).

The compounds described herein can be prepared in a variety of ways known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Variations on the Formula I include the addition, subtraction, or movement of the various constituents as described for each compound. Similarly, when one or more chiral centers are present in a molecule, the chirality of the molecule can be changed. The compounds described herein can be isolated in pure form or as a mixture of isomers. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

Reactions to produce the compounds described herein can be carried out in solvents, which can be selected by one of skill in the art of organic synthesis.

Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

Provided herein are methods of treating or preventing a sexually transmitted infection in a subject. The methods comprise administering to the subject the compounds described herein, wherein the compounds bind the SEVI-fibrils to inhibit the ability of the SEVI-fibrils to enhance a sexually transmitted infection. Compounds contained within International Publication No. WO 2007/011834 are also contemplated herein for use in methods of treating or preventing a sexually transmitted infection.

Also provided are methods of treating or preventing a sexually transmitted infection in a subject. The methods, optionally, comprise identifying a subject with or at risk of developing a sexually transmitted infection and administering to the subject a semen-derived enhancer of viral infection (SEVI)-binding small molecule. The methods can further comprise administering to the subject an anti-viral, an anti-bacterial, or an anti-fungal agent.

The SEVI-binding small molecule can, for example, comprise a hydrophobic molecule, wherein the hydrophobic molecule incorporates into and binds the SEVI-fibrils. SEVI-fibrils are formed as a result of ahydrophobic interactions between component monomer polypeptides. Without meaning to be limited by theory, it is expected that exogenous hydrophobic molecules, such as hydrophobic polypeptides, can be incorporated into and bind the SEVI-fibrils, thus inhibiting the ability of the SEVI-fibrils to interact with the infectious agent causing the sexually transmitted infection. Examples of such hydrophobic molecules include alkanes, oils, fats, and greasy substances in general.

The SEVI-binding small molecule can, for example, comprise an anionic polypeptide supramolecular assembly. Optionally, the anionic supramolecular assembly is water-soluble. Optionally, the anionic supramolecular assembly comprises a soluble hydrogel and other supramolecular assemblies derived from an Ac-(XEXE)n-NH2 (SEQ ID NO:14) polypeptide and related polypeptides. Water-soluble supramolecular assemblies derived from self-assembling anionic polypeptides can, for example, bind to the cationic SEVI fibrils and inhibit interactions between the SEVI-fibrils and the infectious agents. An example of a soluble hydrogel is the PuraMatrix hydrogel. The PuraMatrix hydrogel comprises a (VKVK)n polypeptide fibrillar hydrogel that is not toxic.

The SEVI-binding small molecules can further comprise a bulky side chain, a negatively charged side chain, a coupled moiety, and an anti-viral molecule. A bulky side chain can, for example, comprise a poly-ethylene glycol (PEG) molecule. An anti-viral molecule can, for example, comprise a pradimicin A or AZT molecule.

Also provided are methods of screening for an agent that is capable of binding SEVI-fibrils. Methods of screening for agents that are capable of binding SEVI-fibrils include the steps of providing the agent to be screened, contacting the agent with the SEVI-fibrils, and determining whether the agent to be screened binds the SEVI-fibrils. Binding can be determined, for example, by selecting an assay from the group consisting of a coimmunoprecipitation assay, a colocalization assay, or a fluorescence polarizing assay, as described below. The assays are known in the art, e.g., see Sambrook et al., Molecular Cloning: A Laboratory Manual, 3^{rd} Ed., Cold Spring Harbor Press, Cold Spring Harbor, N.Y. (2001); Dickson, Methods Mol. Biol. 461:735-44 (2008); Nickels, Methods 47(1):53-62 (2009); and Zinchuk et al., Acta Histochem. Cytochem. 40(4):101-11 (2007).

The SEVI-binding agents, SEVI-binding small molecules, anti-viral agents, anti-bacterial agents, anti-fungal agents described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

Such pharmaceutical compositions are optionally, provided in the form of contraceptives or contraceptive agents, such as condoms or spermicides, or lubricants.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are preferably suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, gels and the like. The compounds described herein or derivatives thereof are admixed under sterile conditions with a physiologically acceptable carrier and any preservatives, buffers, or propellants as may be required.

The term pharmaceutically acceptable salt as used herein refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught herein.)

The active compound can be effective over a wide dosage range and is generally administered in a pharmaceutically effective amount. Those of skill in the art will understand that the specific dose level and frequency of dosage for any particular subject may be varied, and it will be understood that the amount of the compound actually administered will usually be determined by a physician, according to the relevant circumstances, including the condition to be treated, the chosen route of administration, the actual compound administered, the age, the activity of the specific compound employed, the metabolic stability and length of action of that compound, the species, age, weight, general health, sex and diet of the subject, the mode and time of administration, rate of excretion, drug combination, and response of the individual subject, the severity of the subject's symptoms, and the like.

### Methods of Treatment or Prevention

The compositions described herein are useful for preventing or reducing the transmission of sexually transmitted infections (STIs). For example, administering a SEVI-binding agent or SEVI-binding small molecule to a subject interferes with the binding of infectious agents to the semen fibrils. Such binding interferes with the infection-enhancing activity of the semen fibrils and prevents or reduces the risk of STIs. The compositions are useful for treatment prior to, during, or after infection. Treatment can completely or partially abolish some or all of the signs and symptoms of transmission of the infection and reduce the likelihood that the treated subject will subsequently develop symptoms of an STI or will delay the onset of symptoms. Thus, for example, treatment can prevent, reduce or delay viral transmission, e.g., HIV transmission.

STIs are infections that can be transferred from one subject to another through sexual contact. In general, STIs are caused by microorganisms that are transmitted via semen, vaginal secretions or blood during sexual contact or by microorganisms that survive on the skin and mucous membranes of the genital area. Sexual contact can include sexual intercourse (vaginal and anal), oral-genital contact, and the use of sexual toys, such as vibrators. Microorganisms transmitted via sexual contact can include, for example, viruses, e.g., HIV, human papilloma virus (HPV), herpesviruses, hepatitis B, and C and cytomegalovirus (CMV); bacteria, e.g., infectious agents responsible for gonorrhea (Neisseria gonorrhoeae); syphilis (Treponema pallidum); chancroid (Haemophilus ducreyi); donovanosis (Granuloma inguinale or Calymmatobacterium granulomatis); lymphogranuloma venereum (LGV) (Chlamydia trachomatis); non-gonococcal urethritis (NGU) (Ureaplasma urealyticum or Mycoplasma hominis); bacterial vaginosis and Staphylococcus aureus; protozoa, e.g., infectious agents responsible for trichommoniasis (Trichomonas vaginalis).

Symptoms of STIs can vary and often the infected subject has no symptoms. However, an asymptomatic subject may be able to pass the disease to a sexual partner. Common symptoms of STI's include, but are not limited to, urethral discharge, genital ulcers, inguinal swellings, scrotal swelling, vaginal discharge, lower abdominal pain, fever, lymphadenopathy (swollen lymph nodes), pharyngitis (sore throat), rash, myalgia (muscle pain), malaise, and mouth and esophageal sores. Both symptomatic and asymptomatic infections can lead to the development of more serious conditions, including AIDS, pelvic inflammatory disease, infertility and tubal (ectopic) pregnancy, genital warts, cervical and other genital cancers.

The compositions and methods are applicable to the transmission of infections by any type of HIV, e.g., HIV-1 and HIV-2. The compositions can be administered to both men and women. The compositions are suitable for a subject who is not infected with HIV, but is at risk for sexually transmitted infection. Subjects who may be at increased risk of becoming infected through sexual contact include those who have unprotected sex, i.e., do not use condoms during sexual intercourse; have multiple sex partners; males who have sexual intercourse with other men; those who have high-risk partner(s), i.e., the sexual partner has multiple sex partners, is a man who has sex with other men, or is an intravenous drug user; or those who have or have recently had a sexually transmitted disease, e.g., syphilis, gonorrhea of chlamydia.

The compositions are also useful in an infected subject, e.g., a subject who has an HIV infection, to reduce the transmission to an uninfected partner. The compositions can be administered to a subject at any stage in the course of HIV infection.

The efficacy of the compositions can be monitored according to standard methods in the art for assessing HIV status, including measuring the level of HIV, using for example a PCR assay, in a clinical sample, e.g., a blood sample, measuring the level of anti-HIV antibodies, using for example, an ELISA or immunoblotting assay, in a clinical sample, e.g., a blood sample, and by monitoring the levels of CD4+ T cells in a clinical sample.

The compositions can be administered in conjunction with other therapeutic or prophylactic modalities to an individual in need of treatment. For example, the compositions may be administered to a subject who practices “safe sex”, i.e., a subject who wears a condom during sexual intercourse or has sexual intercourse with a partner who wears a condom. The condom can be disguised to contain or be coated with the therapeutic agent. The compositions can also be administered in conjunction with other therapies for treating HIV infection, such as standard small molecule pharmaceutical agents, topical microbicides, biopharmaceuticals (e.g., antibodies or antibody-related immunotherapies, siRNAs, shRNAs, antisense oligonucleotides and other RNA inhibitory molecules, microRNAs, and peptide therapeutics), surgery, or in conjunction with any medical devices that may be used to assist the subject. Standard therapy for HIV infection includes highly active antiretroviral therapy, or HAART. Typically, HAART includes a combination (or “cocktail”) of drugs belonging to at least two classes of antiretroviral agents, e.g., a nucleoside analogue reverse transcriptase inhibitors (NARTIs or NRTIs), a non-nucleoside reverse transcriptase inhibitor and a protease inhibitor. Nucleoside reverse transcriptase inhibitors include, for example: AZT (ZDV, zidovudine, Retrovir), ddI (didanosine, Videx), d4T (stavudine, Zerit), 3TC (lamivudine, Epivir), Abacavir (Ziagen), Tenofovir (Viread), Combivir (AZT/3TC combination), Trizivir (AZT/3TC/Abacavir combination), Emtricitabine (FTC, Emtriva), Epzicom (3TC/abacavir combination) and Truvada (tenofovir/emtricitabine combination). Non-nucleoside reverse transcriptase inhibitors (NNRTIs) include, for example: Nevirapine (NVP, Viramune), Delavirdine (DLV, Rescriptor), Efavirenz (EFV, Sustiva, Stocrin) and Etravirine (ETV, Intelence). Protease inhibitors include, for example: Saquinavir (SQV, Invirase), Indinavir (IDV, Crixivan), Ritonavir (RTV, Norvir), Nelfinavir (NFV, Viracept), Amprenavir (APV, Agenerase), Lopinavir (LPV, Kaletra, Aluvia), Atazanavir (ATV, Reyataz), Fosamprenavir (FPV, Lexiva), Tipranavir (TPV, Aptivus) and Darunavir (DRV, Prezista). Other anti-HIV drugs include fusion and attachment inhibitors, including, for example, Enfuvirtide (Fuzeon or T-20) and Maraviroc (MVC, Selzentry, Celsentri); and integrase inhibitor, including for example, Raltegravir (RGV, Isentress). Optionally, the compositions can be incorporated into standard barrier prophylatics, for example male and female condoms.

The duration of treatment with any composition provided herein can be any length of time from as short as one day to as long as the life span of the host (e.g., many years). For example, the composition can be administered once a week (for, for example, 4 weeks to many months or years); once a month (for, for example, three to twelve months or for many years); or once a year for a period of 5 years, ten years, or longer. It is also noted that the frequency of treatment can be variable. For example, the compositions can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

In addition, clinical methods that can assess the degree of a particular disease state can be used to determine if a response is induced. For example blood or laboratory tests may be administered to determine HIV titers before, during and after a course of treatment. The particular methods used to evaluate a response will depend upon the nature of the patient's disorder, the patient's age, and sex, other drugs being administered, and the judgment of the attending clinician.

### Kits

The compositions described herein can also be assembled in kits, together with instructions for use and/or containers, means for administration of the composition, and the like. For example, the kits can include measured amounts of a pharmaceutically acceptable composition including the compounds described herein, and the anti-viral, anti-bacterial, or anti-fungal agents described herein. The instructions for use can be conveyed by any suitable media. For example, they can be printed on a paper insert in one or more languages or supplied audibly or visually (e.g., on a compact disc). The packaging materials can include vials, packets, or intravenous bags, and the kit can also include instruments useful in administration, such as needles, syringes, tubing, condoms, catheters, bandages, and tape. Preferably, the components of the kit are sterile and suitable for immediate use. The invention encompasses kits, however, that include concentrated formulations and/or materials that may require sterilization prior to use.

### Semen Fibrils

The semen fibrils comprise fibrillary aggregates derived from polypeptides in seminal fluid. The fibrillary aggregates can be insoluble fibrous protein aggregates that are generally characterized by a cross-beta sheet quaternary structure; i.e., a monomeric unit contributes a beta strand to a beta sheet, which spans across more than one molecule. The fibrils can be identified using a variety of assays, including fluorescent dyes, e.g., thioflavin T binding, Congo red staining, stain polarimetry, circular dichroism, FTIR or X-ray diffraction analysis. X-ray diffraction analysis reveals characteristic scattering diffraction signals produced at 4.7 and 10.6 Ångstroms (0.47 nm and 1.06 nm), corresponding to the interstrand and stacking distances in beta sheets. The stacks of beta sheet are short and traverse the breadth of the amyloid fibril; the length of the fibril is built by aligned strands.

Semen fibrils can form from semen fibrillary polypeptides or oligomers thereof. A semen fibrillary polypeptide can be a fibril forming fragment of prostatic acid phosphatase (PAP), a protein produced by the prostate and secreted into semen. PAP (also known as ACP-3 or prostatic acid phosphatase precursor 3, ACP3, ACPP or EC 3.1.3.2) is the prostate-specific form of one of five ubiquitous acid phosphatase isozymes that catalyze the conversion of orthophosphoric monoester to alcohol and orthophosphate. PAP is over 100 times more abundant in the prostate that in other tissue types. The cDNA and amino acid sequences encoding a representative human PAP polypeptide (Genbank number NM_{—}001099 [gi:161377405] and NP_{—}001090 [gi:6382064]) are shown as SEQ ID NOs: 1 and 2, respectively. Other amino acid sequences that have been identified for PAP include, without limitation, BAD89417.1, [gi:58737017]; AAB60640, [gi:515997]; AAA60021, [gi:189613]; and NP 064457, [gi:9910502]. Additional amino acid modifications may include PAP-derived sequence derivatives with extensive stretches of hydrophobicity and an associated predilection for fibril formation. The amino acid sequence of human PAP is 386 residues in length; the active form of the enzyme is a homodimer. A peptide corresponding to the amino acid sequence from about residue 248 to about residue 286 in human PAP, i.e.,

(SEQ ID NO: 3)
YGIHKQKEKSRLQGGVLVNEILNHMKRATQIPSYKKLIMY

forms fibrils that enhance the transmission of HIV.

### DEFINITIONS

As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a sexually transmitted infection. The term patient or subject includes human and veterinary subjects.

As used herein the terms treatment, treat, or treating refers to a method of reducing the effects of a sexually transmitted infection or a symptom of the sexually transmitted infection as described above. Thus in the disclosed method, treatment can refer to a 10%, 20%, 30%, 40%, 50%, 60%, 70%, 80%, 90%, or 100% reduction in the severity of a sexually transmitted infection or a symptom of the sexually transmitted infection. For example, a method for treating a sexually transmitted infection is considered to be a treatment if there is a 10% reduction in one or more symptoms of the infection in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the infection or symptoms of the infection.

As used herein, the terms prevent, preventing, and prevention of a sexually transmitted infection as described above refers to an action, for example, administration of a therapeutic agent, that occurs before or at about the same time a subject begins to show one or more symptoms of the disease or disorder, which inhibits or delays onset or exacerbation of one or more symptoms of the infection. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Optional or optionally means that the subsequently described event or circumstance may or may not occur, and that the description includes instances where said event or circumstance occurs and instances where it does not.

Unless defined otherwise, all technical and scientific terms used herein have the same meanings as commonly understood by one of skill in the art to which the disclosed method and compositions belong. Publications cited herein and the material for which they are cited are hereby specifically incorporated by reference in their entireties.

It is understood that the disclosed method and compositions are not limited to the particular methodology, protocols, and reagents described as these may vary.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Administration of Semen Enhancer of Viral Infection (SEVI) to Primary Human Macrophages Stimulates Inflammatory Cytokine Production

Primary human macrophages were prepared from whole blood by Lymphoprep (Accurate Chemical & Scientific; Westbury, N.Y.) density-gradient centrifugation followed by positive selection with CD 14⁺ microbeads (Miltenyi Biotec; Bergisch Gladbach, Germany). The cells were plated in 48-well plates at a concentration of 5×10⁵ cells/mL and differentiated using RPMI-1640 supplemented with 20% fetal bovine serum (FBS) and 5 ng/mL granulocyte macrophage-colony stimulating factor (GM-CSF). After 4 days, the cells were maintained with RPMI-1640 with 20% FBS.

After 7 days, the primary human macrophages were stimulated with either LPS (100 ng/mL), SEVI (10 mM), or both. Cell culture supernatants were collected at 0, 4, and 24-hour timepoints and measurements of TNFα and IL-1β were determined by ELISA. Briefly, 96-well plates were coated with 100 μL/well of capture antibody in coating buffer (eBioscience, Inc.; San Diego, Calif.) and incubated overnight at 4° C. The wells were washed with phosphate buffered saline (PBS) with 0.05% Tween-20 and blocked for 1 hour with 300 μL/well assay diluent (eBioscience, Inc.). 100 μL of the samples (cell culture supernatants) or standards (eBioscience, Inc.) were incubated for 2 hours at room temperature. After washing the wells, 100 μL/well of biotin-conjugated anti-human IL-1β or biotin-conjugated anti-human TNFα detection antibody (eBioscience, Inc.) was added for 1 hour, followed by 100 μL of Streptavidin-HRP (eBioscience, Inc.) for 30 minutes. The wells were developed with TMB (eBioscience, Inc.) and the reaction was stopped with 2N H₂SO₄. Optical density was read at 450 nm with a SpectraCount plate reader (Packard Instrument Company; Meriden, Conn.), and the cytokine levels were then calculated by extrapolation to a standard curve generated using known amounts of recombinant IL-1β and TNFα.

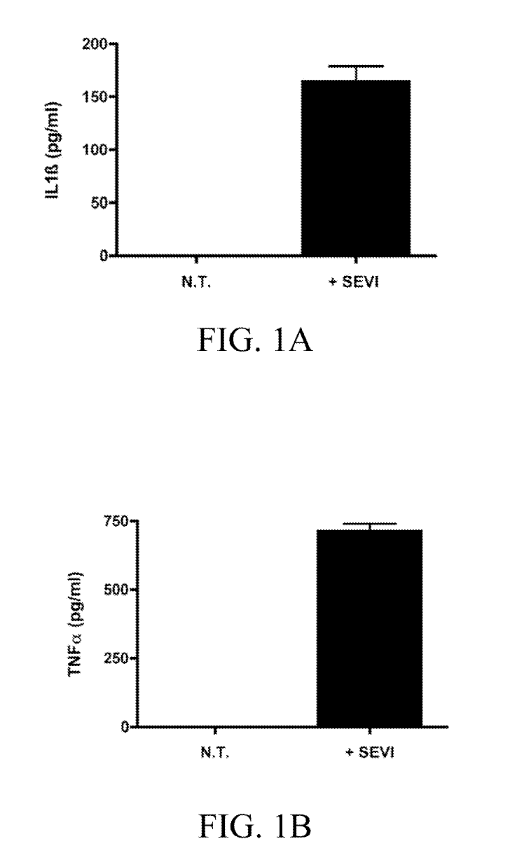

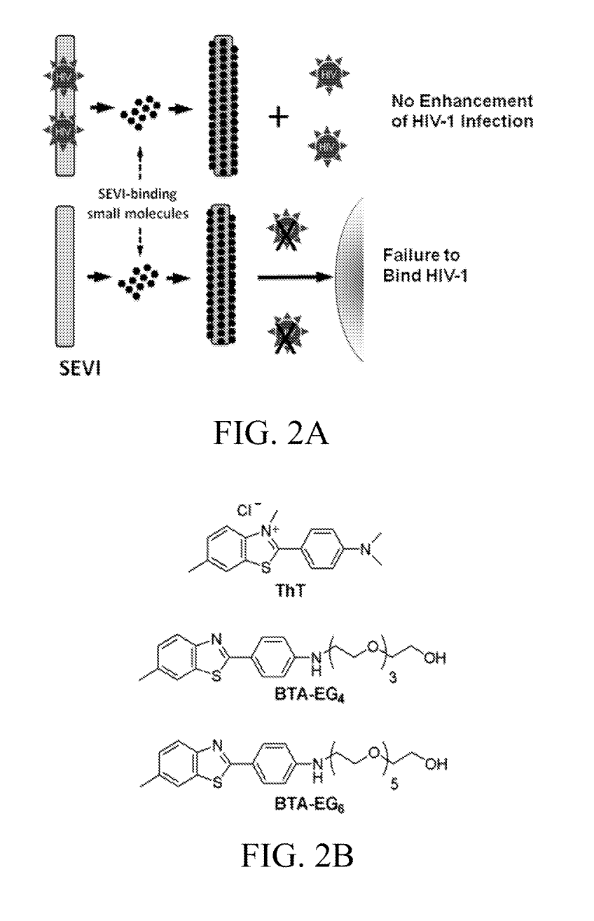

The results are shown in FIG. 1. Addition of SEVI to primary human macrophages as compared to a control elicits an increase in inflammatory cytokine production as evidenced by the increase in IL-1β and TNFα. The results are presented as mean plus or minus the standard error of the mean (SEM) for three independent cell replicates (obtained from a single unit of human blood).

### Example 2

### Identification of Compounds that Bind SEVI and Inhibit SEVI's Effects on HIV Infection

### Synthesis of BTA-EG₆

BTA-EG₆ was synthesized as described previously (Inbar, P., Li, C. Q., Takayama, S. A., Bautista, M. R., and Yang, J. (2006) Chembiochem 7, 1563-1566).

### Cell Culture

CEM-M7 (a gift from N. Landau, New York University, New York, N.Y.) and Jurkat cells were cultured in RPMI 1640 medium supplemented with 10% fetal bovine serum, penicillin (50 units/ml), and streptomycin (50 μg/ml). SiHa cells were cultured in DMEM medium supplemented with 10% fetal bovine serum, penicillin (50 units/ml), and streptomycin (50 μg/ml). A2En cells (a gift from S. Greene, Louisiana State University Health Sciences Center, New Orleans, La.), and 3EC1 cells (a gift from R. Pyles, University of Texas Medical Branch, Galveston, Tex.) were cultured in keratinocyte serum-free medium (Invitrogen, Carlsbad, Calif.) supplemented with bovine pituitary extract (50 mg/liter), recombinant epidermal growth factor (5 μg/liter), CaCl₂ (44.1 mg/liter), and Primocin (0.1 mg/ml). PBMCs were isolated from whole blood by Lymphoprep density gradient centrifugation. PBMCs were stimulated for 48 h in RPMI 1640 medium supplemented with 5% human IL-2 (ZeptoMetrix, Buffalo, N.Y.), 5 μg/ml PHA (Sigma, St. Louis, Mo.), 20% fetal bovine serum, penicillin (50 units/ml), and streptomycin (50 μg/ml).

### SEVI and Semen

PAP248-286 and biotinylated PAP248-286, in which a biotin was added to the amino terminus of the peptide, was synthesized and dissolved in PBS at a concentration of 10 mg/ml. Fibrils were formed by agitation in an Eppendorf Thermomixer at 1400 rpm (Eppendorf, Hauppauge, N.Y.) and 37° C. for 72 h. Semen samples were obtained from the Strong Fertility Center (Rochester, N.Y.) and Fairfax CryoBank (Fairfax, Va.). Samples were pooled, aliquoted, and stored at −80° C.

### Fluorescence Polarization

100 μg/ml SEVI was mixed with 16 μg/ml FITC-heparin and concentrations of BTA-EG₆ ranging from 0 to 200 μg/ml. Samples were incubated 1 h at room temperature and read on a PerkinElmer Life Sciences Envision 2012 multilabel reader (PerkinElmer, Waltham, Mass.) at an excitation λ=480 nm and emission λ=535 nm. The horizontal and vertical polarized fluorescence intensities were recorded, and the calculated polarization was determined in millipolarization units.

### Determination of the Binding Affinity of BTA-EG6 and SEVI Fibrils

Binding of BTA-EG₆ to SEVI fibrils was measured according to the centrifugation assay described by Levine (LeVine, H., 3rd (2005) Amyloid 12: 5-14) for BTA-1 to Aβ fibrils. Briefly, 200 μl of various concentrations of BTA-EG₆ in PBS were incubated in the presence or absence of 10 μg of SEVI fibrils to give a final volume of 220 μl of solution. These incubations were performed in duplicate runs and allowed to equilibrate overnight at room temperature. After equilibration, each solution was centrifuged at 16,000×g for 30 min. The supernatants were separated from the pelleted fibrils, and 220 μl of fresh PBS was added to resuspend the pellets. A 100 μl aliquot of each resuspended pellet was pipetted into a cuvette (ultra-microcuvette, 10-mm light path, Hellma®, Müllheim, Germany), and the fluorescence of BTA-EG₆ was determined at 355 nm excitation and 430 nm emission using a spectrofluorometer (Photon Technology International, Inc., Birmingham, N.J.). Error bars represent standard deviations from the mean. The graph was plotted and fitted using the following one-site specific binding algorithm to determine Kd: Y=Bmax×X/(Kd+X), where X is the concentration of BTA-EG₆, Y is the specific binding fluorescence intensity, and Bmax corresponds to the apparent maximal observable fluorescence upon binding of BTA-EG6 to SEVI fibrils. The data were processed using Origin 7.0 (MicroCal Software, Inc., Northampton, Mass.).

### Flow Cytometry

10⁵ Jurkat cells were incubated with biotinylated SEVI fibrils (40 μg/ml) with and without BTA-EG6 at a concentration of 10 (low) or 30 μg/ml (high) or heparin (100 μg/ml) as a positive control for interfering with SEVI binding to the cell surface. Cells were incubated for 1 h at 37° C., washed, and stained for 1 h with a covalent conjugate of streptavidin and fluorescein isothiocyanate (SA-FITC). Cells were washed and run on an Accuri C6 Flow Cytometer (Accuri Cytometers, Ann Arbor, Mich.). Data were analyzed using FlowJo (TreeStar Inc, Ashland, Oreg.).

### Infectivity Assays

For infection of CEM-M7 cells, X4 tropic HIV-1_{IIIB} (21 ng/ml p24) or R5 tropic HIV-1_{ADA} (60 ng/ml p24) was pretreated for 10 min at room temperature with 15 μg/ml SEVI in the presence or absence of BTA-EG₆. Treated virions were then added to 5×10⁴ CEM-M7 cells/well in 96-well flat-bottomed tissue culture plates. After 2 h, the medium axis were replaced. Infection was assayed after 48 h by quantifying luciferase expression using the Promega Dual-Luciferase assay and a Beckman Coulter DTX880 plate reader.

For infections using semen, pooled human semen samples were added to virions at a 1:1 dilution and incubated for 15 min at room temperature in the presence or absence of BTA-EG₆. After 15 min, the semen and virus mixture was diluted 1:15 into 5×10⁴ CEM-M7 cells/well in a 96-well plate. Cells were washed after 1 h, and infection was assayed at 48 h as above.

For infections of PBMCs, R5 tropic HIV-1BaL preincubated with 15 μg/ml SEVI in the presence or absence of BTA-EG6 was added to 2×105 PHA/IL-2-stimulated PBMCs/well in 96-well flat-bottomed tissue culture plates. Cells were washed at 3 h, and infection was analyzed at day 4 using the HIV-1 p24 antigen capture assay (Advanced Bioscience Laboratory).

### Virus Binding Assay

HIV-1 IIIB or ADA virions were pretreated with 15 μg/ml SEVI and added to 5×10⁴ Jurkat cells, or A2En cells, in the presence or absence of BTA-EG6. After 90 min, cells were washed to remove any unbound virus, and bound virions were detected using an HIV-1 p24 antigen capture assay (Advanced Bioscience Laboratory).

### Cytokine and Chemokine Studies

HIV-1 BaL virions were pretreated with 15 μg/ml SEVI and added to 5×10⁴ A2En cells in the presence or absence of BTA-EG6. Supernatant was collected at 6 and 24 h, and the production of the chemokines IL-8 and Mip-3α was measured by ELISA (R&D Systems). To assess semen-mediated chemokine production, SiHa cells were treated with semen, as described above, in the presence or absence of BTA-EG6. After 6 h, supernatants were collected, and the production of the chemokines IL-8 and Mip-3α was measured by ELISA (R&D systems, Minneapolis, Minn.).

### Toxicity Studies

The cervical epithelial cell lines SiHa, A2En (endocervical), and 3EC1 (ectocervical) were treated for 12 h with BTA-EG₆ at concentrations up to 66 μg/ml, 10 times the IC₅₀. After 12 h, cell viability was analyzed by measuring cellular metabolic activity using the resazurin cytotoxicity assay, AlamarBlue® (Invitrogen), in accordance with the manufacturer's protocol. Cytokine and chemokine production was assessed at 12 h by ELISA (R&D systems). Cells were also treated with 0.1% nonoxynol-9 as a positive control for cytotoxicity and with 0.1 μg/ml FSL1, a synthetic diacylated lipoprotein derived from Mycoplasma salivarium (InvivoGen, San Diego, Calif.), as a positive control for chemokine production.

SEVI fibrils are highly cationic, with a negative charge of +6.5 at neutral pH and +8 at pH 5, as would be seen in the vaginal mucosa. The cationic nature of SEVI is required for its ability to enhance HIV infection. This suggests that SEVI acts in a manner similar to other cationic polymers to enhance HIV infectivity.

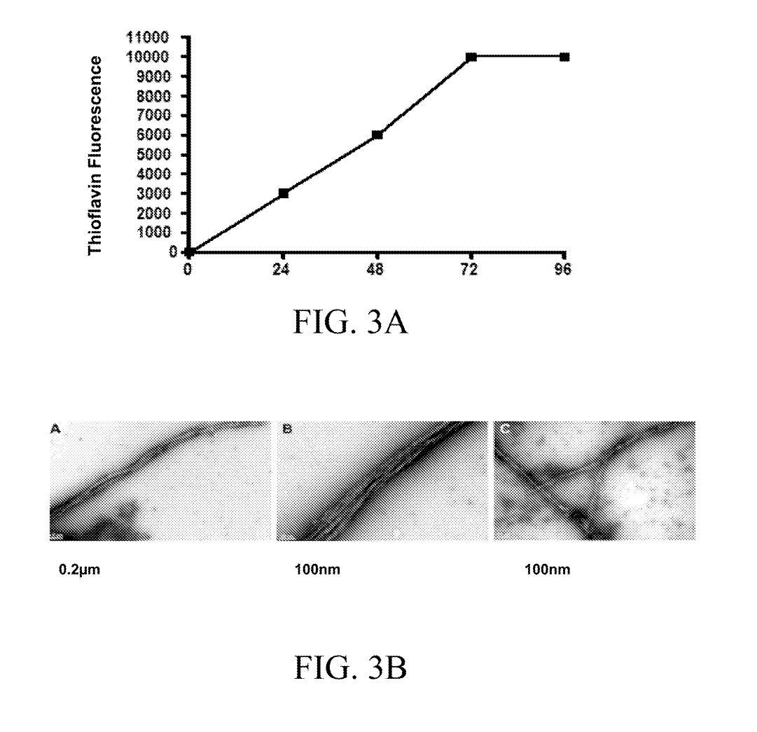

To find compounds that bind SEVI fibrils and inhibit the ability of SEVI to enhance HIV infection, a model system of SEVI fibrils was developed. Fragments of prostatic acid phosphatase (PAP) from amino acid 248 to amino acid 286 were found to form SEVI fibrils. The PAP248-286 fragments at a concentration 10 mg/ml in PBS were agitated at 37° C. and 1400 RPM to form fibrils (FIG. 3A). The SEVI fibrils were viewed by electron microscopy at 72 hours (FIG. 3B).

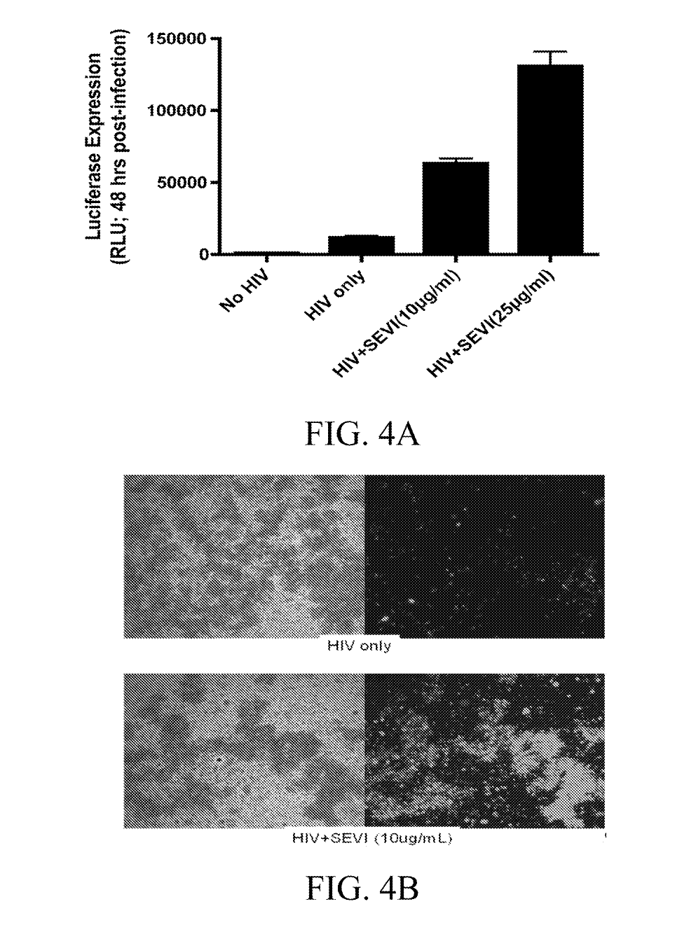

To determine if the SEVI fibrils could enhance HIV-1 infection, CEM 5.25 cells were exposed to infectious HIV-1 for 2 hours in the presence or absence of SEVI. It was found that increasing concentrations of SEVI enhanced HIV-1 infection as evidenced by the increase in luciferase activity (FIG. 4A). Further, an increase in GFP expression indicative of HIV-1 infectivity was observed in cells treated with SEVI (FIG. 4B).

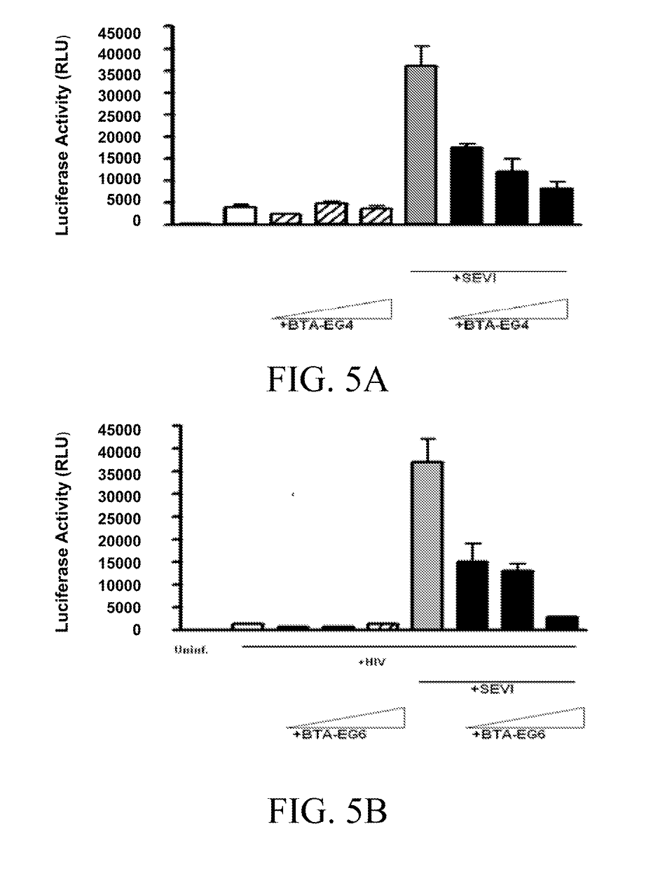

To determine if BTA-EG₄ and BTA-EG₆ were able to inhibit SEVI mediated enhancement of HIV infection, CEM 5.25 cells were exposed to infectious HIV-1 for 2 hours in the presence or absence of SEVI fibrils. In the presence of SEVI fibrils, increasing concentrations of BTA-EG₄ and BTA-EG₆ resulted in decreasing levels of luciferase activity (FIGS. 5A and 5B), indicating a decrease in the ability of SEVI fibrils to enhance HIV-1 infection.

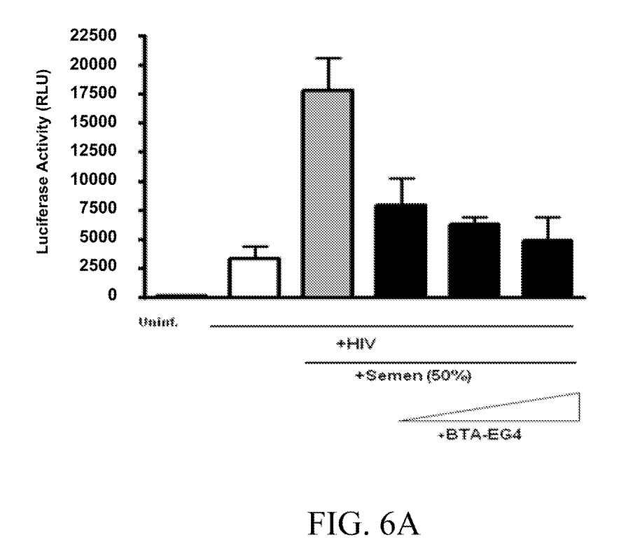

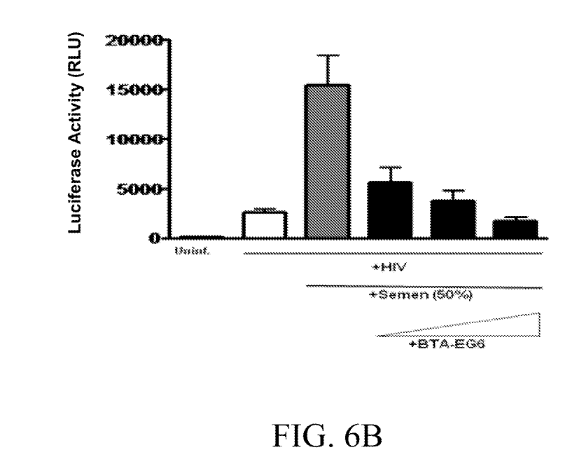

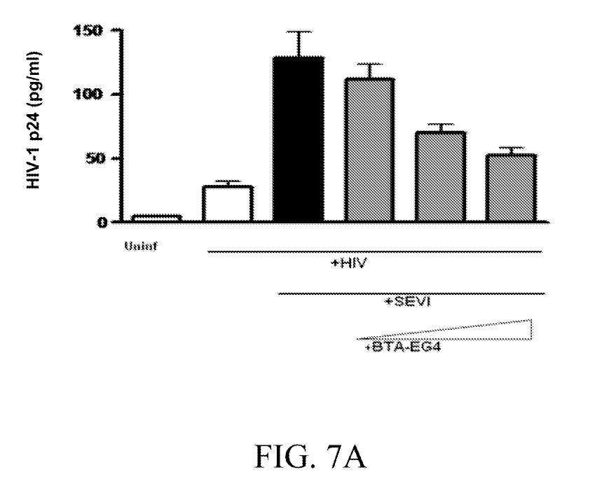

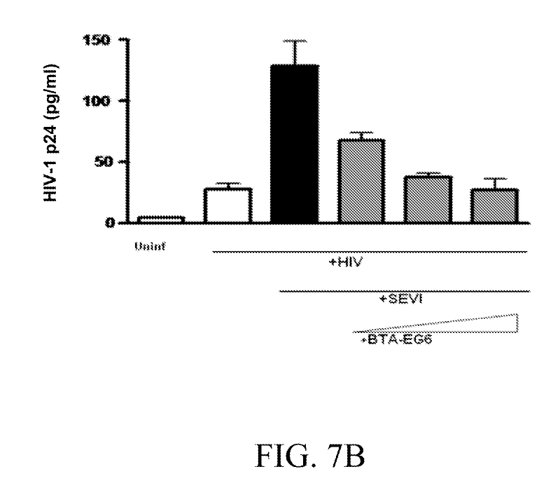

To determine if BTA-EG₄ and BTA-EG₆ were capable of inhibiting semen mediated enhancement of HIV infection, HIV-1 IIIB virions were preincubated with 50% semen and increasing concentrations of BTA-EG₄ and BTA-EG₆. After 10 minutes, the stocks were diluted 15 fold and incubated with CEM 5.25 cells. The increasing concentrations of BTA-EG₄ and BTA-EG₆ resulted in a decrease in luciferase activity (FIGS. 6A and 6B), indicating that BTA-EG₄ and BTA-EG₆ were capable of inhibiting semen mediated enhancement of HIV infection. It was further found that BTA-EG₄ and BTA-EG₆ were capable of inhibiting SEVI-enhanced binding of HIV to the cell surface. HIV-1 IIIB virions were pretreated with 10 μg/mL SEVI and added to Jurkat cells with or without increasing concentrations of BTA-EG₄ and BTA-EG₆. After 90 minutes cells were washed to remove any unbound virus and bound virions were detected using a p24 ELISA. Increasing concentrations of BTA-EG₄ and BTA-EG₆ resulted in a decrease in HIV-1 binding to the cells (FIGS. 7A and 7B).

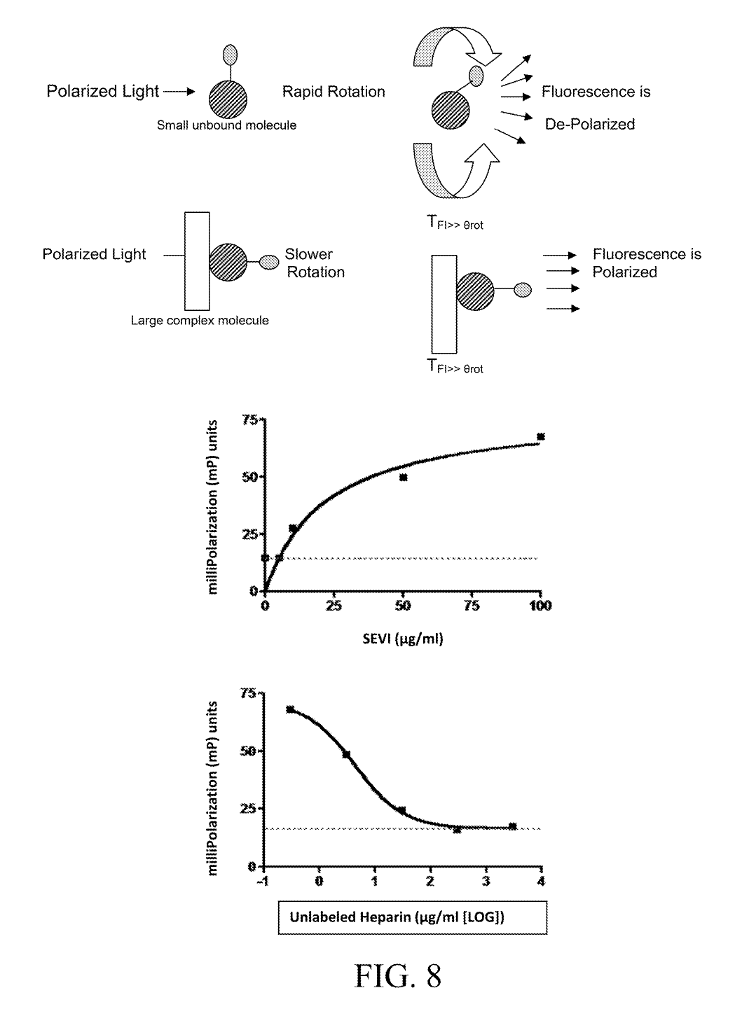

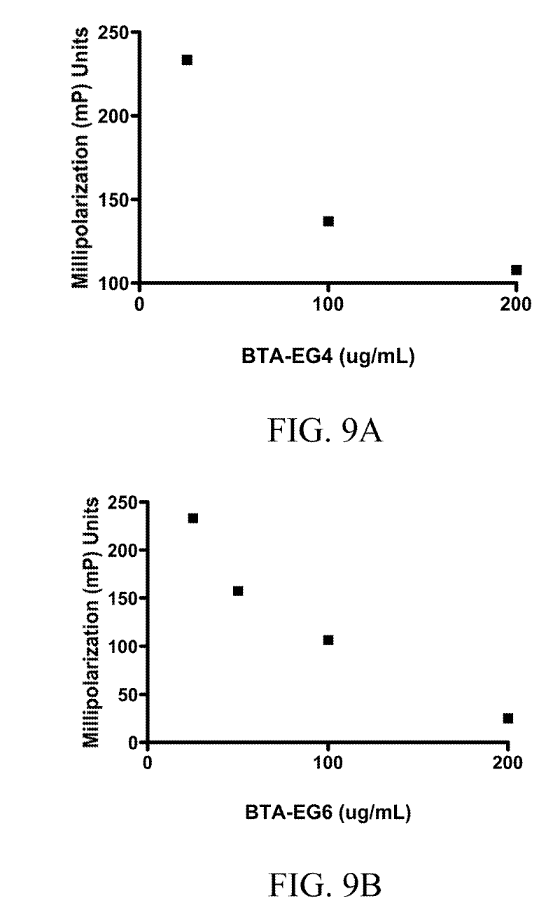

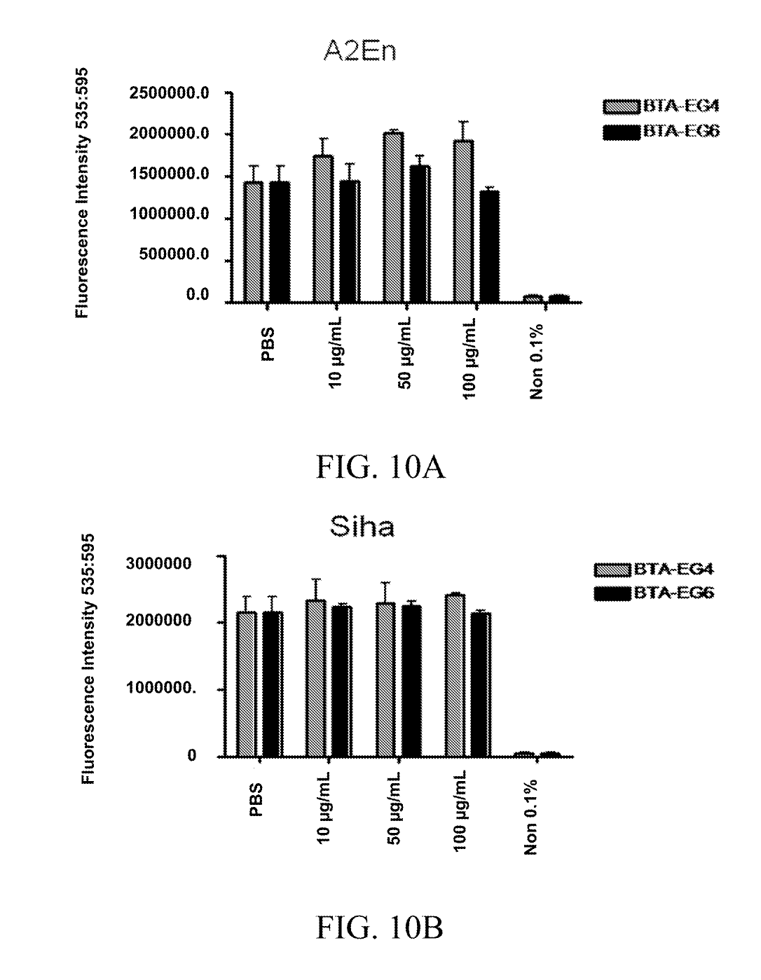

In order to find other small molecules that bind SEVI, a fluorescence polarizing screen was developed. The screen is shown in FIG. 8. Polarized light passed over a small unbound molecule with a fluorescent moiety will produce rapid rotation and will result in fluorescence that is de-polarized. Polarized light passed over SEVI bound to a small molecule with a fluorescent moiety will result in fluorescence that is polarized. As a proof of principle, SEVI fibrils were diluted to concentrations ranging from 5 to 100 μg/ml in the presence of 16 mg/ml of FITC-heparin. Samples were incubated at excitation of λ=480 and emission λ=535 (FIG. 8, left graph). When unlabeled heparin was added to the SEVI-FITC-heparin mixture, the polarization decreased as evidenced in FIG. 8, right graph. Using this assay, it was shown that BTA-EG₄ and BTA-EG₆ were also capable of binding SEVI fibrils. 100 mg/ml of SEVI was mixed with FITC-heparin in varying concentrations of BTA-EG₄ and BTA-EG₆. Samples were incubated for 1 hour at room temperature and polarized fluorescence was measured. It was shown that both BTA-EG₄ and BTA-EG₆ were capable of binding SEVI as evidenced by decreasing levels of polarized light (FIGS. 9A and 9B).

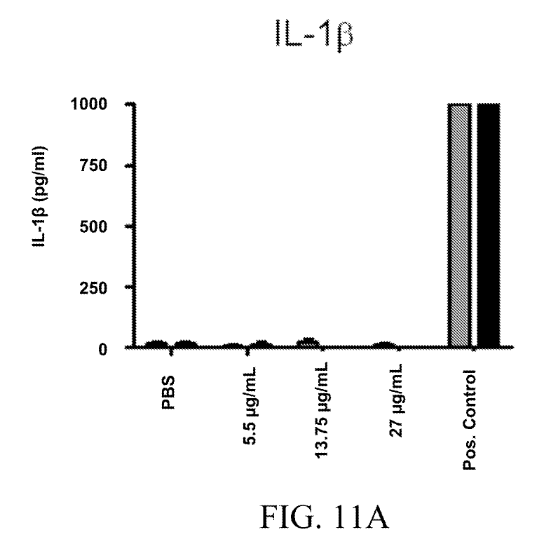

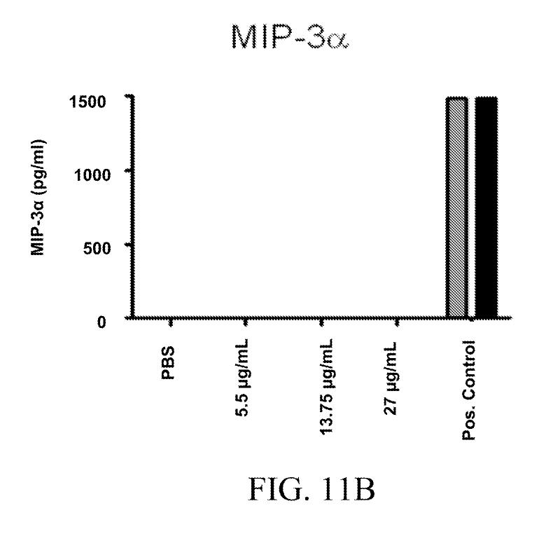

To determine if BTA-EG₄ and BTA-EG₆ were capable of being administered to cervical epithelial cells without cytotoxicity, the cervical cell lines A2En (endocervical) and SiHa cells were treated with BTA-EG₄ and BTA-EG₆ for 12 hours at concentrations up to 10 times greater than the inhibitory concentration. At 12 hours, viability was measured with Alamar Blue and it was shown that BTA-EG₄ and BTA-EG₆ did not affect the cell viability of A2En and SiHa cells. It was further shown in SiHa cells that BTA-EG₄ and BTA-EG₆ do not induce cytokine production. SiHa cells were treated with BTA-EG₄ and BTA-EG₆ at varying concentrations for 6 hours and cytokine production was examined by ELISA. As shown in FIGS. 11A-11C, IL-1b, MIP-3a, and TNF-a levels were unaffected by varying concentrations of BTA-EG₄ and BTA-EG₆.

### The Thioflavin-T Analog BTA-EG₆ Binds SEVI Fibrils

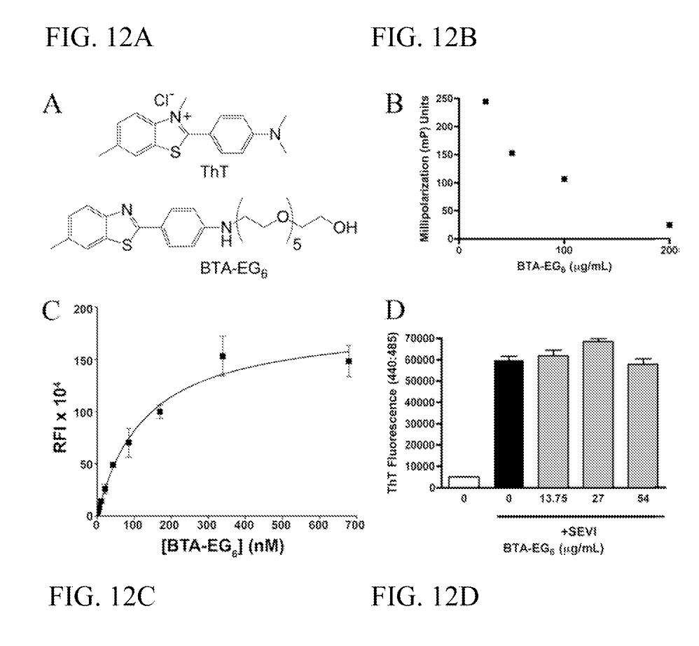

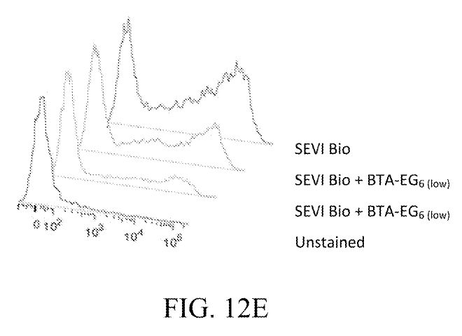

ThT is able to intercalate into the generic β-sheet structure of amyloid fibrils. The benzothiazole aniline derivative, BTA-EG₆, is a ThT analog carrying a hexa(ethylene glycol) moiety (FIG. 12A). This molecule binds to Aβ fibrils and interferes with the ability of Aβ-binding proteins to interact with the fibrils. Fluorescence polarization was used to measure the ability of BTA-EG₆ to bind SEVI. Increasing concentrations of BTA-EG₆ were added to 50 μg/ml SEVI that had been preincubated with 16 μg/ml FITC-heparin, a known SEVI binder. BTA-EG₆ was able to displace fluorescent heparin from the SEVI fibrils in a dose-dependent fashion (FIG. 12B), thus showing an interaction between these molecules and the fibrils.

Having observed an interaction between these molecules, the binding of BTA-EG₆ to SEVI fibrils was assessed by quantifying its binding affinity. A fluorescence-based assay was used to determine the Kd between BTA-EG₆ and the SEVI fibrils (see LeVine Amyloid 12(3): 12-15 (2005). FIG. 12C shows the relative fluorescence intensity (RFI) of BTA-EG₆ bound to SEVI fibrils as a function of exposure of the SEVI peptides to increasing concentrations of BTA-EG₆. Fitting the data in FIG. 12C with a one-site specific binding algorithm revealed a value of Kd=127±22 nM (R²=0.98). For comparison, this same fluorescence binding assay was used to measure the affinity of BTA-EG₆ for binding to aggregated Alzheimer disease-related Aβ(1-42) peptides, which gave a value of Kd=111±32 nM (R2=0.95); this value was similar to the Kd value for binding of BTA-EG₆ to SEVI fibrils.

To examine whether the interaction of BTA-EG₆ with SEVI impacted the stability of the fibrils, preformed SEVI fibrils were incubated with BTA-EG₆ for 3 h. After that time, fibrillar structures were examined by ThT fluorescence. ThT changes in fluorescence intensity when intercalated into the β-sheet structure common to amyloid fibrils; therefore, ThT fluorescence serves as a surrogate measure for fibrillar structure of SEVI and for the stability of SEVI fibrils. As seen in FIG. 12D, the addition of BTA-EG₆ had no effect on fibrillar stability as measured by ThT. Unlike in the case with ThT, the fluorescence intensity of BTA-EG₆ does not change upon binding to amyloid fibrils. The intrinsic fluorescence of BTA-EG₆, therefore, does not interfere with the analysis of fibril stability using this assay. To further explore the interactions between SEVI fibrils and BTAEG6, the binding of this compound to SEVI was tested to determine if it could inhibit the ability of the fibrils to interact with the negatively charged surface of mammalian cells. Jurkat T-cells were incubated with 35 μg/ml SEVI-biotin fibrils, which were formed by fibrillization of a biotinylated PAP248-286 peptide, in the presence of 5.5 or 13 μg/ml BTA-EG₆. Heparin was used as a positive control as this polyanionic compound has been shown to inhibit the binding of SEVI fibrils to the cell surface. Binding of the fibrils to the cell surface was detected using SA-FITC. As seen in FIG. 12E and Table 1, increasing concentrations of BTA-EG₆ inhibited the ability of SEVI fibrils to interact with and bind the cell surface, as measured by both the percentage of cells with bound fibrils and the mean fluorescence intensity of the cells. Table 1 shows that BTA-EG₆ binding to SEVI inhibits interaction of SEVI fibrils with the cell surface. Jurkat cells were incubated with SEVI-biotin (SEVI-Bio) for 1 h in the presence or absence of 5.5 (low) or 27 μg/ml (high) BTA-EG₆. Surface-bound fibrils were detected with SA-FITC and measured by flow cytometry. Results are shown as percentage of cells with bound fibrils (SA-FITC+) as well as mean fluorescent intensity (MFI).

TABLE 1

| | SA-FITC+ | |
|---|---|---|
| Sample | % | MFI |
| Unstained | 1.65 | 28 |
| SEVI-Bio | 48.1 | 2525 |
| SEVI-Bio + BTA-EG₆ (low) | 36.6 | 95.3 |
| SEVI-Bio + BTA-EG₆ (high) | 21.2 | 38.7 |
| SEVI-Bio + heparin | 31.7 | 376 |

### BTA-EG₆ Inhibits SEVI-Mediated Enhancement of HIV-1 Infection

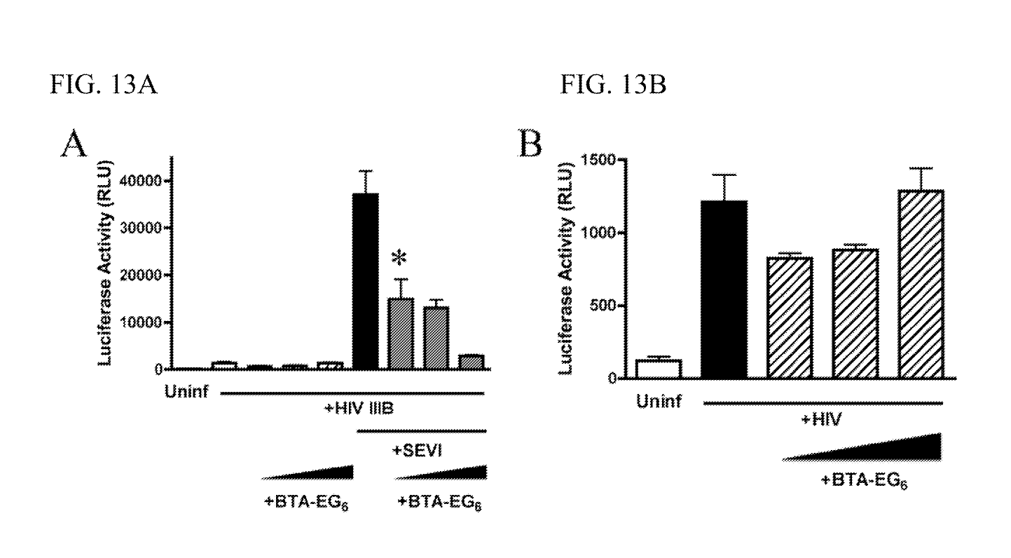

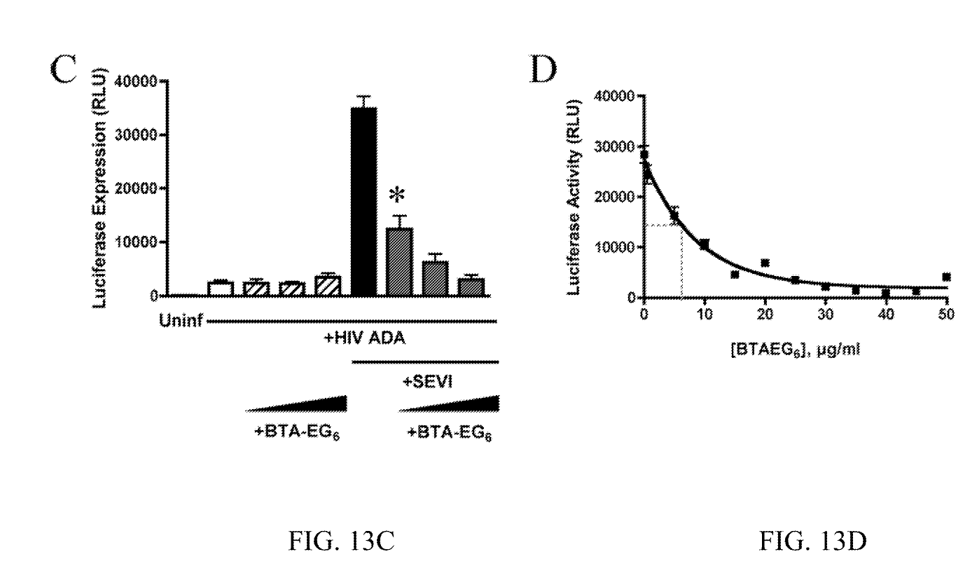

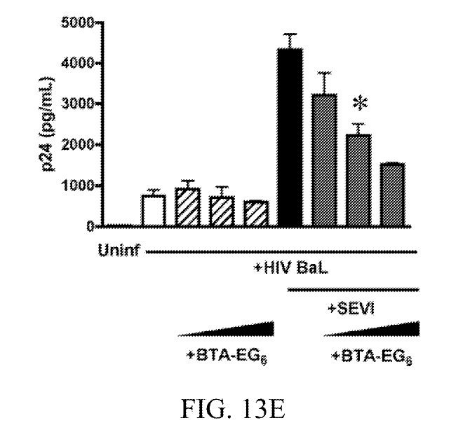

Having observed that BTA-EG₆ was able to inhibit the interaction of SEVI with the cell surface, whether it could effectively inhibit SEVI-mediated enhancement of HIV-1 infection was investigated. CEM-M7 cells were infected with HIV-1 strain IIIB plus 15 μg/ml SEVI fibrils in the presence of increasing concentrations of BTA-EG₆. CEM-M7 cells are a CD4⁺ CCR5⁺ CXCR4⁺T/B cell hybrid cell line and contain HIV LTR-driven luciferase and GFP reporter gene cassettes. The HIV LTR is a weak transcriptional regulator in the absence of its cognate, virally encoded trans-activator, Tat. As a result, luciferase and GFP expression levels in these cells are directly responsive to HIV-1 infection; this property therefore provides a convenient method to determine the extent of viral infection. BTA-EG₆ was able to effectively inhibit SEVI-mediated enhancement of HIV infection in a dose-dependent fashion, reducing infectivity nearly back to baseline levels (i.e. levels detected in the absence of SEVI) at the highest concentrations tested (FIG. 13A). Importantly, BTA-EG₆ had no effect on the infectivity of HIV virus alone, even at the highest concentrations (FIG. 13B), indicating that this effect was not due to direct inhibition of intrinsic virus infectivity.

Because most sexually transmitted HIV-1 infections are the result of R5 viruses, whether the effect of BTA-EG₆ extended to a well characterized R5 strain was examined. CEM-M7 cells were infected with HIV-1ADA and 15 μg/ml SEVI, with and without increasing concentrations of BTA-EG₆. Once again, BTA-EG₆ showed a significant dose-dependent inhibition of SEVI-mediated enhancement of HIV-1 infection (FIG. 13C). No effect on the infectivity of HIV-1ADA was observed in the absence of SEVI (FIG. 13C). The IC₅₀ of the BTA-EG₆ for inhibition of SEVI-mediated enhancement of HIV-1 infection was also determined. To do this, CEM-M7 cells were infected with HIV-1ADA and 15 μg/ml SEVI in the presence of BTA-EG₆. Ten different BTA-EG₆ concentrations were tested, ranging from 0.4 to 50 μg/ml. The data were fit to an exponential decay curve to calculate the IC₅₀, and results are shown in FIG. 13D. The calculated IC₅₀ was 6.6 μg/ml for BTA-EG₆ (equivalent to 13 μM).

Next, PBMCs were infected with HIV-1BAL with 15 μg/ml SEVI in the presence or absence of increasing concentrations of BTA-EG₆. BTA-EG₆ was able to inhibit SEVI-mediated enhancement of HIV-1 infection in PBMCs at similar concentrations to those seen in other cell lines (FIG. 13E). BTA-EG₆ had no effect on the infectivity of HIV-1BaL in PBMCsin the absence of SEVI (FIG. 13E). Thus, the effects of BTA-EG₆ are neither strain-dependent nor cell type-dependent, and the compound has no effect on HIV-1 infection in the absence of SEVI.

### BTA-EG₆ Inhibits Semen-Mediated Enhancement of HIV-1 Infection

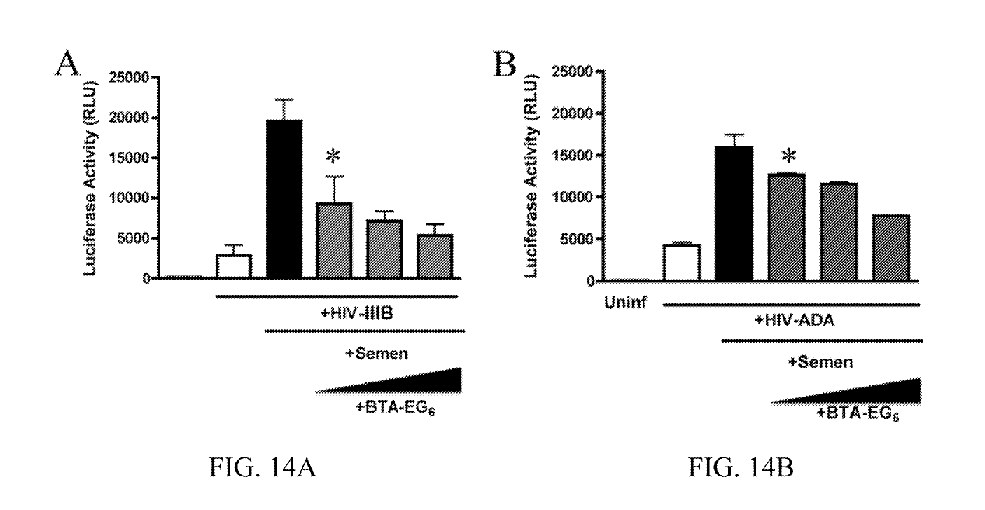

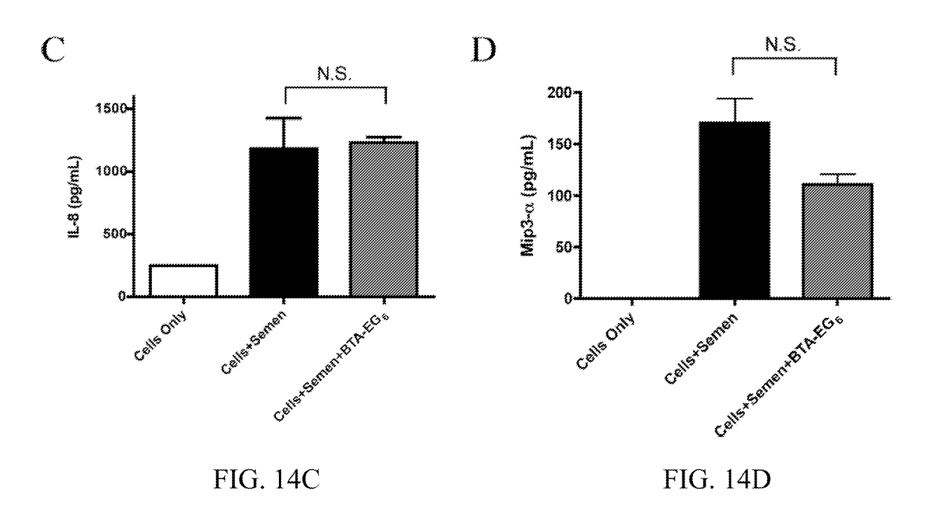

For BTA-EG₆ to be a viable microbicide candidate, it must be effective not just against the effects of SEVI but should be able to effectively inhibit the infection-enhancing activity of human semen. Therefore, the effect of this compound on semen-mediated enhancement of HIV-1 infection was examined. As human semen has been reported to be toxic to cultured cells, a protocol that minimized this toxicity was used. Pooled human semen was added to HIV-1IIIB virus in a 1:1 dilution. After 15 min, this solution was added to cells at a 1:15 dilution for a final concentration of 3.3%. As shown in FIG. 14A, BTA-EG₆ efficiently inhibited the semen mediated enhancement of HIV-1 infection at similar concentrations to those active against SEVI alone. FIG. 14B shows that this effect extended to infection with an R5 virus, HIV_{ADA} as well.

A follow-up experiment was performed to test whether the effects of BTA-EG₆ on semen were specific to the infection enhancing components in semen (i.e. SEVI) or due to a more general inhibitory effect against semen. To do this, BTA-EG₆ inhibited semen-mediated chemokine release was examined. Human semen can be pro-inflammatory, mediating the release of IL-8 and MIP-3α from cervical endothelial cells. This property is thought to be due to the presence of several pro-inflammatory mediators but is not due to the presence of SEVI as SEVI does not stimulate the release of IL-8 or MIP-3α. SiHa cells, a cervical endothelial cell line, was treated with pooled human semen with or without 33 μg/ml BTA-EG₆, a dose five times higher than the IC₅₀. After 6 h, supernatants were collected and analyzed for production of IL-8 or MIP-3α. Pooled human semen effectively elicited the production of these chemokines from SiHa cells as expected, whereas BTA-EG₆ had no inhibitory effect on semen-stimulated chemokine production (FIGS. 14C and D).

### BTA-EG₆ Inhibits SEVI-Mediated Attachment of HIV-1 to the Cell Surface

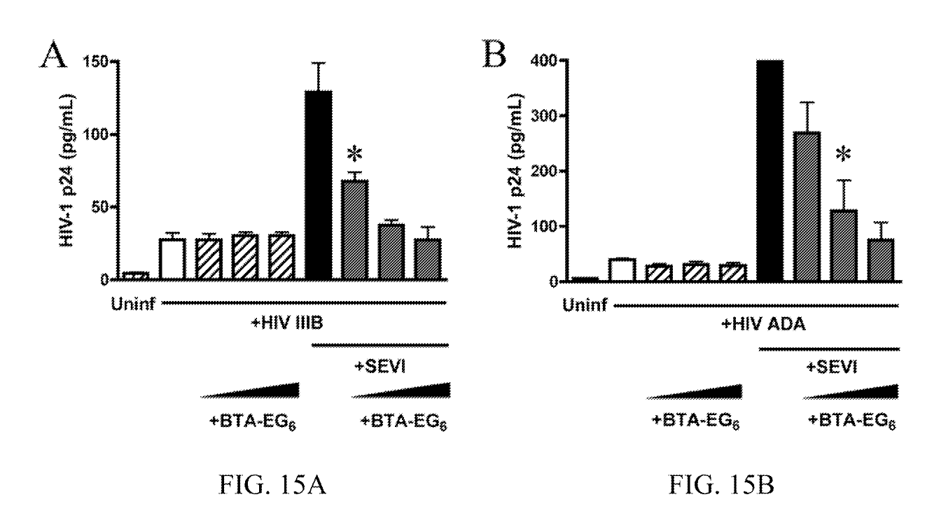

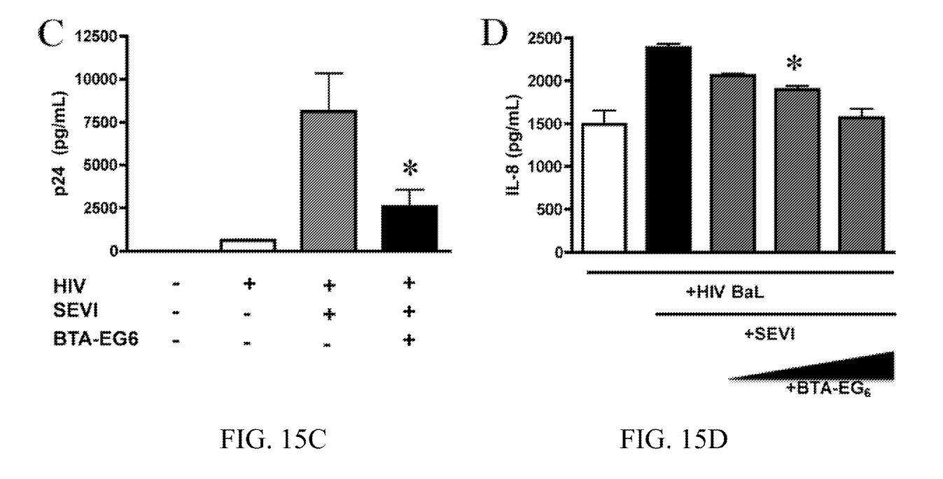

To more closely examine how BTA-EG₆ mediates its inhibitory effects on SEVI-mediated HIV-1 infection enhancement, the ability of this compound to interfere with SEVI-enhanced binding of HIV-1 virions to the cell surface was examined. The cationic nature of SEVI enhances the binding of virions to the cell surface, which allows it to neutralize the electrostatic repulsion between the negatively charged HIV-1 virion and target cell surface. Jurkat T cells were incubated with HIV-1IIIB virions and 15 μg/ml SEVI in the presence or absence of increasing concentrations of BTA-EG₆. Surface-bound virions were then measured by p24 ELISA after washing off unbound virus. SEVI was able to strongly enhance the binding of virions to the cell surface, and this effect was efficiently abrogated by BTAEG₆ (FIG. 15A). BTA-EG₆ had no effect on the binding of HIV virions to the cell surface in the absence of SEVI (FIG. 15A). Similar results were obtained with an R5 virus, HIV-1ADA (FIG. 4B). Additionally, this experiment was performed using A2En cells, a primary cell-derived endocervical cell line. It was found that SEVI also enhanced binding of virions to the surface of these cervical epithelial cells and that this effect was inhibited by BTA-EG₆ (FIG. 15C).

Whether SEVI would increase HIV-1-mediated chemokine production and whether BTA-EG6 could inhibit this effect was also tested. HIV stimulates the release of MIP-3 and IL-8 from vaginal and cervical epithelial cells. Because SEVI increases the interactions between virions and epithelial cells, SEVI likely increases HIV-mediated chemokine release as well. Therefore, A2En cells were exposed to HIV-1_{BAL} virions with and without SEVI, in the presence or absence of BTA-EG₆. As seen in FIG. 4D, SEVI modestly increased the release of IL-8 from cells treated with virus, and BTA-EG₆ was able to inhibit this release. Similar results were also obtained for MIP-3α.

### BTA-EG₆ is not Toxic to Cervical Cells

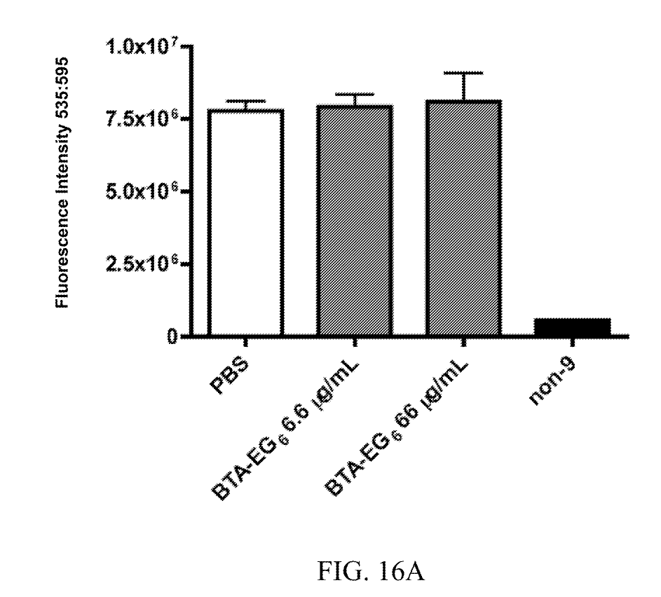

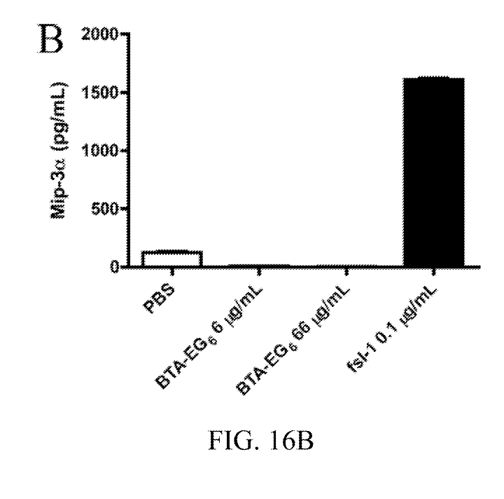

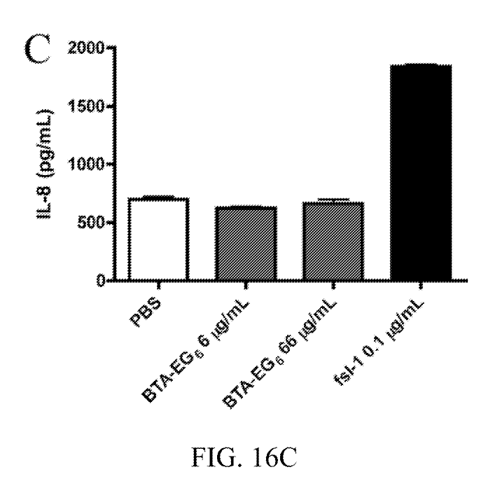

For a compound to be a legitimate HIV-1 microbicide candidate, it must not have toxic or inflammatory effects on the cervical endothelium. Loss of this protective layer leads to an increased ability for HIV-1 to cross the mucosal barrier, and inflammatory effects drive recruitment of HIV-1 target cells, further decreasing the natural barriers against successful transmission of HIV. Therefore, the effects of BTA-EG6 on cervical endothelial cells were examined. To do this, the following cell lines were used: 1) SiHa cells, a cervical carcinoma cell line; 2) A2En cells, a primary cell-derived line from the endocervical endothelium; and 3) 3EC1 cells, a primary cell-derived line from the ectocervical endothelium. To evaluate the effects of BTA-EG₆ on cell viability, the compound was added to cells at concentrations up to 10× the IC₅₀ for up to 24 h. Viability was assessed at 24 h by using the resazurin cytotoxicity assay. Resazurin cytotoxicity data were confirmed by trypan blue counts of viable cells. FIG. 16A shows that BTA-EG₆ did not have any effects on cell viability, even at the highest concentrations tested.

Nonoxynol-9 (non-9), a spermicide, was used as a positive control. Whether treatment with BTA-EG₆ led to the production of inflammatory cytokines and chemokines from the cervical cell lines was examined. All three cervical cell lines were treated for 6 h with concentrations of BTA-EG₆ ranging from 6.6 to 66 μg/ml. Cell culture supernatants were then assessed for the presence of the inflammatory cytokines and chemokines Mip-3α (FIG. 5B), IL-8 (FIG. 16C), IL-1β, and TNF-α. These cytokines and chemokines were selected because they are up-regulated by other candidate microbicides and because they may play a role in microbicide-mediated enhancement of HIV-1 infection. BTA-EG₆ did not lead to the release of any of these cytokines or chemokines, even at the highest doses tested. These results indicate that BTA-EG₆ is not toxic to cervical endothelial cells.

BTA-EG₆ inhibited SEVI-mediated enhancement of infection by both X4 (HIV-1_{IIIB}) and R5 (HIV-1_{ADA}) strains, in a dose-dependent fashion. In the case of HIV-1_{ADA}, the IC50 was 13 μM; this value is 100-fold higher than the measured Kd of BTA-EG₆ for binding to aggregated SEVI peptides (127 nM). Without being limited by theory, one explanation for this difference is that the ability of BTA-EG₆ to compete with virion/fibril or virion/cell interactions requires a greater number of BTA-EG₆ molecules than the noncompetitive binding of BTA-EG₆ to SEVI alone. BTA-EG₆ also inhibited SEVI-enhanced infection of primary cells (human peripheral blood mononuclear cells) in a dose-dependent fashion, and it blocked SEVI-enhanced binding of X4 (HIV-1_{IIIB}) and R5 (HIV-1_{ADA}) strains to target cells (including both Jurkat T cells and A2En endocervical cells). These data showed that (i) SEVI enhances the ability of HIV-1 virions to elicit IL-8 and MIP-3α from A2En endocervical cells and (ii) this can be inhibited by BTA-EG6. Without being limited by theory, these data show that BTA-EG₆ and related compounds not only reduce the efficiency of HIV-1 infection of target cells but also reduce the level of target cell recruitment to virus-exposed genital mucosal tissue. BTA-EG6 effectively prevents semen mediated enhancement of HIV infectivity, showing that this activity of semen can be targeted by specifically inhibiting the SEVI fibrils. BTA-EG6 did not inhibit other properties of semen, such as the ability to elicit pro-inflammatory chemokines Thus, BTA-EG6 is an effective microbicide target

### Example 3

### Characterization of Monomeric and Oligomeric Binding to SEVI Fibrils

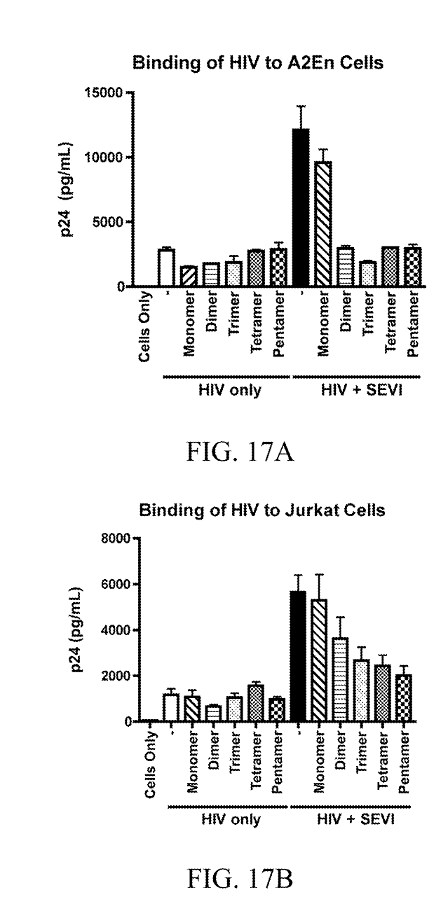

HIV-1 IIIB virions were pretreated with 15 μg/ml SEVI and added to 5×10⁴ A2En cells (immortalized primary human endocervical cells) (FIG. 17A) or to Jurkat T cells (a CD4+ human T cell line) (FIG. 17B) in the presence or absence of test compound BTA-EG₆ in monomeric, dimeric, trimeric, tetrameric or pentameric forms (at a final concentration of 25 μM). After 90 min, cells were washed to remove any unbound virus, and bound virions were detected using an HIV-1 p24 antigen capture assay (Advanced Bioscience Laboratory, Rockville, Md.). The data showed reduced HIV-1 p24 antigen capture in the presence of SEVI as compared to capture in the absence of SEVI.

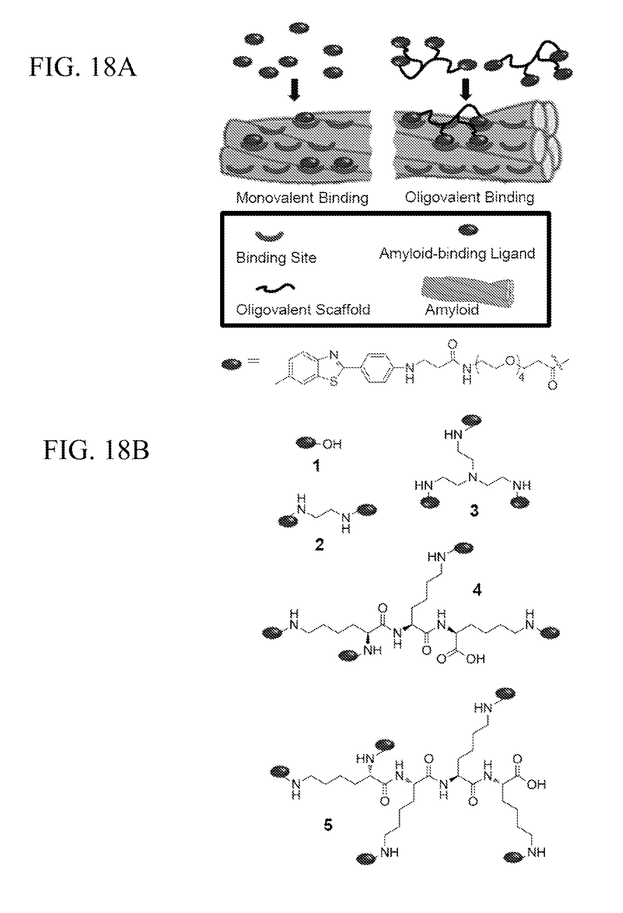

These data suggest that the oligovalent scaffold may influence binding. FIG. 18 shows the structure of a benzothiazole aniline (BTA)-based monomer (1), dimer (2), trimer (3), tetramer (4), and pentamer (5) and a schematic of how the scaffold may affect binding. To test whether binding affinity/avidity is reduced in oligomeric forms, the binding of monomeric and oligomeric forms was assayed to determine the affinity/avidity constants (K_{d}) for the binding of monomer and oligomers 1-5 to amyloid fibrils formed from SEVI peptides. Affinity/avidity constants were estimated using a standard fluorescence assay, as described recently (J. S. Olsen et al, J. Biol. Chem., 2010, 285, 35488-35496). The BTA based monomer has a higher K_{d} than oligomeric forms. Estimated K_{d}s are show in Table 2.

TABLE 2

| | SEVI Fibrils |
|---|---|
| Compound | K_{d} (nM) |
| Monomer (1) | 107 ± 16 |
| Dimer (2) | 75 ± 10 |
| Trimer (3) | 40 ± 6 |
| Tetramer (4) | 56 ± 6 |
| Pentamer (5) | 84 ± 21 |

1. A method of treating or preventing a sexually transmitted infection in a subject, the method comprising administering to the subject a semen-derived enhancer of viral infection (SEVI)-binding agent, wherein the agent comprises a compound of the following formula: 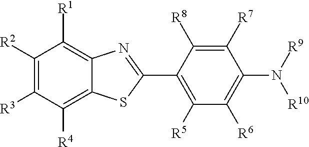
or a pharmaceutically acceptable salt or prodrug thereof, wherein:
R¹, R², R³, R⁴, R⁵, R⁶, R⁷, and R⁸ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl; and
R⁹ and R¹⁰ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, or substituted or unsubstituted heteroaryl.

2. The method of claim 1, wherein R³ is methyl.

3. The method of claim 1, wherein R¹, R², R⁴, R⁵, R⁶, R⁷, and R⁸ are hydrogen.

4. The method of claim 1, wherein R⁹ is 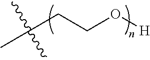
wherein n is an integer from 0 to 20.

5. The method of claim 4, wherein n is 4 or 6.

6. (canceled)

7. The method of claim 1, wherein R¹⁰ is hydrogen.

8. The method of claim 1, wherein the compound is

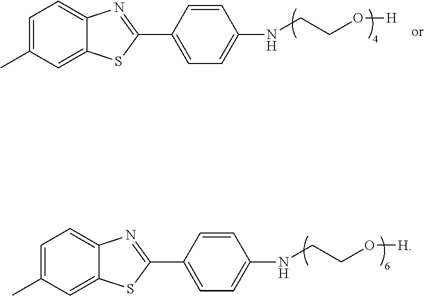

9. (canceled)

10. The method of claim 1, further comprising administering to the subject an anti-viral, an anti-bacterial, or an anti-fungal agent.

11. The method of claim 1, wherein the sexually transmitted infection is selected from the group consisting of a viral infection, a bacterial infection, and a fungal infection.

12. (canceled)

13. (canceled)

14. (canceled)

15. A method of treating or preventing a sexually transmitted infection in a subject, the method comprising administering to the subject a semen-derived enhancer of viral infection (SEVI)-binding small molecule, wherein the SEVI-binding small molecule comprises a hydrophobic molecule, wherein the hydrophobic molecule incorporates into and binds the SEVI-fibrils.

16. The method of claim 15, further comprising administering to the subject an anti-viral, an anti-bacterial, or an anti-fungal agent.

17. The method of claim 15, wherein the sexually transmitted infection is selected from the group consisting of a viral infection, a bacterial infection, and a fungal infection.

18. (canceled)

19. (canceled)

20. (canceled)

21. A method of treating or preventing a sexually transmitted infection in a subject, the method comprising administering to the subject a semen-derived enhancer of viral infection (SEVI)-binding small molecule, wherein the SEVI-binding small molecule comprises an anionic polypeptide supramolecular assembly.

22. The method of claim 21, further comprising administering to the subject an anti-viral, an anti-bacterial, or an anti-fungal agent.

23. The method of claim 21, wherein the sexually transmitted infection is selected from the group consisting of a viral infection, a bacterial infection, and a fungal infection.

24. (canceled)

25. (canceled)

26. (canceled)

27. The method of claim 21, wherein the anionic polypeptide supramolecular assembly is water-soluble.

28. The method of claim 21, wherein the anionic polypeptide supramolecular assembly comprises a soluble hydrogel and other supramolecular assemblies derived from an Ac-(XEXE)n-NH2 (SEQ ID NO:14) polypeptide and related polypeptides.

29. The method of claim 15, wherein the SEVI-binding small molecule further comprises a bulky side chain, a negatively charged side chain, a coupled moiety, and an antiviral molecule.

30. The method of claim 29, wherein the bulky side chain is poly-ethylene glycol.

31. The method of claim 29, wherein the antiviral molecule comprises pradimicin A or AZT.

32. A pharmaceutical composition comprising:
(a) a first agent, wherein the agent comprises a SEVI-binding agent comprising a compound of the following formula: 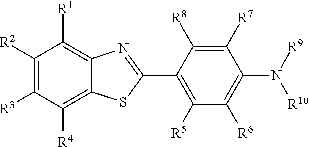
or a pharmaceutically acceptable salt or prodrug thereof, wherein:
R¹, R², R³, R⁴, R⁵, R⁶, R⁷, and R⁸ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl; and
R⁹ and R¹⁰ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, or substituted or unsubstituted heteroaryl; and
(b) a second agent selected from the group consisting of an anti-viral, an anti-bacterial, or an anti-fungal agent.

33. The pharmaceutical composition of claim 32, wherein R³ is methyl.

34. The pharmaceutical composition of claim 32, wherein R¹, R², R⁴, R⁵, R⁶, R⁷, and R⁸ are hydrogen.

35. The pharmaceutical composition of claim 32, wherein R⁹ is 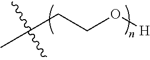
wherein n is an integer from 0 to 20.

36. The pharmaceutical composition of claim 35, wherein n is 4 or 6.

37. (canceled)

38. The pharmaceutical composition of claim 32, wherein R¹⁰ is hydrogen.

39. The pharmaceutical composition of claim 32, wherein the compound is

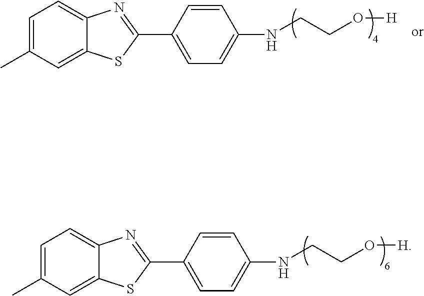

40. (canceled)

41. A pharmaceutical composition comprising:
(a) a first agent, wherein the first agent comprises a semen-derived enhancer of viral infection (SEVI)-binding small molecule, wherein the SEVI-binding small molecule comprises a hydrophobic molecule, wherein the hydrophobic molecule incorporates into and binds the SEVI-fibrils.
(b) a second agent selected from the group consisting of an anti-viral, an anti-bacterial, and an anti-fungal agent.

42. A pharmaceutical composition comprising:
(a) a first agent, wherein the agent comprises a semen-derived enhancer of viral infection (SEVI)-binding small molecule, wherein the SEVI-binding small molecule comprises an anionic polypeptide supramolecular assembly.
(b) a second agent selected from the group consisting of an anti-viral, an anti-bacterial, and an anti-fungal agent.

43. The pharmaceutical composition of claim 42, wherein the anionic polypeptide supramolecular assembly is water-soluble.

44. The pharmaceutical composition of claim 42, wherein the anionic polypeptide supramolecular assembly comprises a soluble hydrogel and other supramolecular assemblies derived from an Ac-(XEXE)n-NH2 (SEQ ID NO:14) polypeptide and related polypeptides.

45. The pharmaceutical composition of claim 41, wherein the SEVI-binding small molecule further comprises a bulky side chain, a negatively charged side chain, a coupled moiety, and an antiviral molecule.

46. The pharmaceutical composition of claim 45, wherein the bulky side chain is poly-ethylene glycol (PEG).

47. The pharmaceutical composition of claim 45, wherein the antiviral molecule comprises pradimicin A or AZT.


SEQUENCE LISTING (3 sequences)

SEQ ID NO: 1 - DNA, 1161 bp, Homo sapiens
atgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaactcatcatgtattctgcgcatgacactactgtgagtggcctacagatggcgctagatgtttacaacggactccttcctccctatgcttcttgccacttgacggaattgtactttgagaagggggagtactttgtggagatgtactatcggaatgagacgcagcacgagccgtatcccctcatgctacctggctgcagccccagctgtcctctggagaggtttgctgagctggttggccctgtgatccctcaagactggtccacggagtgtatgaccacaaacagccatcaaggtactgaagacagtacagattag

SEQ ID NO: 2 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 3 - protein, 40 aa, Homo sapiens
YGIHKQKEKSRLQGGVLVNEILNHMKRATQIPSYKKLIMY